(19) United States
(12) Patent Application Publication
(10) Pub. No.: US 2017/0159115 A1
(43) Pub. Date: Jun. 8, 2017

(54) SINGLE MOLECULE NUCLEIC ACID SEQUENCING WITH MOLECULAR SENSOR COMPLEXES

(71) Applicant: Stratos Genomics, Inc., Seattle, WA (US)

(72) Inventors: Mark Stamatios Kokoris, Bothell, WA (US); Robert N. McRuer, Mercer Island, WA (US); John C. Tabone, Kirkland, WA (US); Cara Machacek, Seattle, WA (US)

(21) Appl. No.: 15/232,593

(22) Filed: Aug. 9, 2016

Related U.S. Application Data
(60) Provisional application No. 62/203,308, filed on Aug. 10, 2015.

(51) Int. Cl. C12Q 1/68; G01N 27/447; G01N 33/487

(52) U.S. Cl. CPC C12Q 1/6869; G01N 33/48721; G01N 27/44791

(57) ABSTRACT

The present disclosure relates to methods and constructs for single molecule electronic sequencing of template nucleic acids. The constructs are molecular sensor complexes which comprise a processive nucleic acid processing enzyme localized to a nanopore. Conformational changes in the enzyme induced by single nucleic acid processing events are transduced into electric signals by the nanopore, which are used to identify individual nucleotides. The methods can include the steps of providing a membrane with the nanopore and the enzyme complexed with a template nucleic acid localized proximal to an opening in the pore, contacting the enzyme with an ion conductive reaction mixture including the reagents required for nucleic acid processing, providing a voltage drop across the pore that induces ion current through the pore that is modulated by conformational changes in the enzyme, measuring current through the pore over time to detect nucleotide-dependent conformational changes in the enzyme, and identifying the type of nucleotide processed by the enzyme using current modulation characteristics, thus determining sequencing information about the nucleic acid molecule.

### BACKGROUND

Technical Field

This disclosure relates generally to evaluation of nucleic acids by enzymes that catalyze reactions having nucleic acids as their reactants or products. More specifically this disclosure relates to sequencing nucleic acids, the activity of evaluation by polymerases or other enzymes, or combinations thereof.

Description of the Related Art

The genome of an organism provides a blueprint for life that encodes all information forming the basis of development, function, and reproduction. Determining the nucleic acid sequences of complete genomes has the potential to provide useful tools for basic research into how and where organisms live, as well as in applied sciences, such as drug development. In clinical medicine, sequencing tools can be used for diagnosis and to develop treatments for a variety of pathologies, including cancer, heart disease, autoimmune disorders, multiple sclerosis, or obesity. An individual's unique DNA sequence provides valuable information concerning susceptibility to certain diseases and enables screening for early detection and receipt of preventative treatment. Furthermore, given a patient's individual genetic blueprint, clinicians will be capable of administering personalized therapy to maximize drug efficacy and to minimize the risk of an adverse drug response. Similarly, determining the blueprint of pathogenic organisms can lead to new treatments for infectious diseases and more robust pathogen surveillance. Thus, whole genome DNA sequencing will likely contribute to the foundation of modern medicine. However, the day when an individual can review a copy of his or her own personal genome with a doctor to determine appropriate choices for a healthy lifestyle or a proper course of treatment for a presenting disease has not yet arrived.

Sequencing of a diploid human genome requires determining the sequential order of approximately 6 billion nucleotides. The ability to decipher the blueprint is slowly improving through improvements in nucleic acid sequencing technologies. However, to date only a few human genomes have been sequenced. First the time and cost for determining genomic sequences must come down to a level that large genetic correlation studies can be carried out by scientists. Furthermore, the technology must reach the point that it is accessible to virtually anyone in a clinical environment regardless of economic means and personal situation.

The first generation of sequencing technology, often referred to as “Sanger sequencing,” was originally developed by Frederick Sanger in 1977. This technique uses sequence specific termination of DNA synthesis and fluorescently modified nucleotide reporter substrates to derive sequence information. These samples require some molecular amplification such as polymerase chain reaction (PCR) to produce a fluorescent signal for reliable detection. The method sequences a target nucleic acid strand, or read length, of up to 1000 bases long by using a modified reaction in which sequencing is randomly interrupted at select base types (A, C, G or T) and the lengths of the interrupted sequences are determined by capillary gel electrophoresis. The length then determines what base type is located at that length. Many overlapping read lengths are produced and their sequences are overlaid using data processing to determine the most reliable fit of the data. The Sanger method was used to provide most of the sequence data in the Human Genome Project, which published the first complete sequence of the human genome in 2001. This project took over 10 years and nearly $3 B to complete.

Commercial “second generation” DNA sequencing tools emerged in 2005 in response to the low throughput of first generation methods. To address this problem, second generation sequencing tools, also referred to as “next generation sequencing”, exploit molecular amplification of target DNA and massively parallelized chips, including arrays of microbeads (Roche and Life Technologies/Thermo Fisher Scientific), DNA nanoballs (Complete Genomics), and DNA clusters (Illumina). In most second generation tools, tens of thousands of identical amplified strands are anchored to a given location to be read in a process consisting of successive flushing and scanning operations. The “flush and scan” sequencing process involves sequentially flushing in reagents, such as labeled nucleotides, incorporating nucleotides into the DNA strands, stopping the incorporation reaction, washing out the excess reagent, scanning to identify the incorporated base and finally treating that base so that the strand is ready for the next “flush and scan” cycle. This cycle is repeated until the reaction is no longer viable. Due to the large number of flushing, scanning and washing cycles required, the time to result for second generation methods is generally long, often taking days. This repetitive process also limits the average read length produced by most second-generation systems under standard sequencing conditions to approximately 35 to 400 bases. Other disadvantages to second generation sequencing include complex sample preparation, amplification-related variation in sequence equality with regard to representation bias and accuracy, the dephasing of the signal readout due to signal reduction and increased inhomogeneity with read length increases, the need for many samples to justify machine operation, and significant data storage and interpretation requirements. Together, first and second generation sequencing technologies have led to a number of scientific advances. However, given the inherent limitations of these technologies, researchers still have not been able to unravel the complexity of whole genomes.

Technologies to sequence DNA at the single molecule level, i.e., “third-generation” sequencing, have been anticipated to resolve most, if not all, of the above problems. In these approaches, the error-prone amplification step is eliminated during sample preparation. One single molecule sequencing strategy that has generated much interest to date is based on the use of nanopores. The basic concept of nanopore sequencing is to pass a single-stranded DNA molecule through a nanoscale pore embedded in a membrane and measure the ensuing changes in ion current passing through the pore. In theory, individual bases induce characteristic electronic signals as they pass through the narrowest constriction of the pore, generating nucleotide-specific signals. The head-to-tail sequential feed-through of DNA should allow for unlimited read length without complicated amplification or labeling steps. In practice, nanopore-based sequencing has been hampered by the fast translocation speed of DNA through nanopores together with the fact that several nucleotides contribute to the recorded signals in the most developed systems, limiting resolution of the read-out and preventing single base calling.

One strategy to overcome these technical challenges exploits the DNA handling properties of nucleic acid processing enzymes to control the rate of DNA translocation through the nanopore. In one example, the MinION sequencer commercialized by Oxford Nanopore Technologies employs a DNA handling enzyme as a motor to ratchet single-stranded DNA, base by base, through a modified nanopore. Although this system succeeds in slowing DNA translocation to a speed compatible with sequencing, it is still unable to directly associate current levels with individual nucleotides. To address these issues, base-calling algorithms are necessary to deconvolute sequencing reads. Moreover, this system cannot resolve sequences in stretches of homopolymers longer than its read window of ˜4 bases. To date, the error rate inherent in this nanopore-based system still is too high to achieve reliable de novo whole genome assembly.

Alternative approaches to single molecule sequencing have been proposed and developed in which the activity of DNA polymerase is monitored in real-time. One such “sequencing by synthesis” (SBS) system has been commercialized by Pacific Biosciences in the SMRT sequencing platform, which directly observes the processive DNA polymerization activity of a single surface-tethered DNA polymerase enzyme. Nucleotide incorporation events are detected in real-time as fluorescent probes are released from each of the four uniquely labeled dNTPs upon formation of the phosphodiester bond. Detection of liberated fluorescent probes relies on “zero mode waveguide” nanostructure arrays, which provide optical observation volume confinement, enabling single-fluorophore detection despite relatively high labeled dNTP concentrations. Although capable of delivering long sequencing reads, the SMRT platform has high single read error rate and requires high cost optical instrumentation, precluding it as a practical sequencing solution for the majority of users at present.

Other real-time sequencing strategies based on fluorescent detection are disclosed in US patent application no. 2011/0312529 to Illumina and U.S. Pat. No. 8,911,972 to Pacific Biosciences. In these approaches, conformational changes in the DNA polymerase protein itself are monitored as the enzyme binds and releases specific nucleotide substrates during chain elongation. Conformational changes are detected by FRET using DNA polymerases labeled with fluorescent label and quencher probes. Base calling may be based on the timing of incorporation events, or additional fluorescent signals emitted from incorporated nucleotides. All the aforementioned methods based on single-molecule fluorescent detection suffer the same disadvantages of photobleaching and low sensitivity that leads to poor signal-to-noise and high error rate.

As such, direct sequencing of DNA by detection of its constituent parts has yet to be achieved in a high-throughput process due to the small size of the nucleotides in the chain (about 4 Angstroms center-to-center) and the corresponding signal to noise and signal resolution limitations therein. While significant advances have been made in the field of DNA sequencing, there continues to be a need in the art for new and improved methods. The present invention fulfills these needs and provides further related advantages.

### BRIEF SUMMARY

The invention is generally directed to methods, constructs, and systems for sequencing nucleic acids. The constructs are herein referred to as “molecular sensor complexes” and function to transduce single nucleotide processing events into electrical signals that are used to identify the nucleotides. In particular, the invention is directed to real time single molecule sequencing of nucleic acids using molecular sensor complexes comprising current-conducting transmembrane pores and conformationally flexible nucleic acid processing enzymes. The methods, constructs, and systems of the present invention provide considerable advantages over the current generation of sequencing technologies in that they require no target amplification or labeling steps during sample preparation and benefit from the superior sensitivity of electronic detection.

In one aspect, the invention provides a method for determining sequence information about a nucleic acid molecule including the steps of: i) providing a membrane having at least one transmembrane pore with a top opening and a bottom opening, and having a single processive nucleic acid processing enzyme localized proximal to one of the openings and complexed with a nucleic acid; ii) contacting the processive nucleic acid processing enzyme with an ion conductive reaction mixture including reagents required for nucleic acid processing by the enzyme; iii) providing a voltage differential that induces ion current through the pore, wherein the ion current is only substantially modulated by nucleotide-dependent conformational changes in the processive nucleic acid processing enzyme; iv) measuring the current through the transmembrane pore over time to detect nucleotide-dependent conformational changes in the processive nucleic acid processing enzyme; and v) identifying the type of nucleotides processed by the processive nucleic acid processing enzyme using current modulation characteristics, thus determining sequence information about the nucleic acid molecule.

In some embodiments, the current modulation characteristics may include the magnitude of the current through the transmembrane pore or the shape of the measured current through the transmembrane pore over time.

In other embodiments, the transmembrane pore may be a protein. In yet other embodiments, the protein may be αHL, MspA, or OmpG. In a further embodiment, the current modulation characteristics may be changes to the spontaneous OmpG current gating activity.

In some embodiments, the processive nucleic acid processing enzyme is a DNA polymerase. In further embodiments, the DNA polymerase may be Klenow fragment, Phi29, or DPO4. In yet other embodiments, the nucleic acid is a primed single stranded template and the reaction mixture includes reagents required for polymerase mediated DNA synthesis. In yet other embodiments, the conformational changes are produced by binding of single nucleotides and incorporation into a growing strand by the DNA polymerase. In further embodiments, the sequencing reaction includes four different types of nucleotides or nucleotide analogs, each corresponding to the bases A, G, C, and T or A, C, G, and U. In further embodiments, each of the types of nucleotides or nucleotide analogs produces a different conformational change in the polymerase enzyme. In yet further embodiments, the different conformational changes may be structurally or temporally distinct or have different current blockage levels. In another embodiment, the step of contacting the DNA polymerase with an ion conductive reaction mixture including reagents required for nucleic acid processing includes the steps of sequentially flooding the DNA polymerase with mixtures including each single nucleotide.

In other embodiments, the processive nucleic acid processing enzyme is a DNA exonuclease which may be a native or an engineered enzyme with exonuclease activity. In some embodiments, the nucleic acid is a double-stranded or single-stranded nucleic acid and the reaction mixture includes reagents required for exonuclease mediated nucleic acid degradation. In yet other embodiments, the binding and release of single nucleotides from the nucleic acid produces the nucleotide-dependent conformational changes in the exonuclease. In further embodiments, each of the types of nucleotides produces a different conformational change in the exonuclease enzyme. In yet further embodiments, the different conformational changes may be structurally or temporally distinct or have different current blockage levels.

In other embodiments, the processive nucleic acid processing enzyme is a DNA helicase which may be a native or an engineered enzyme with helicase activity. In some embodiments, the nucleic acid is a double-stranded nucleic acid and the reaction mixture includes reagents required for helicase mediated nucleic acid strand separation. In other embodiments, the breaking of hydrogen bonds between individual pairs of nucleotides produces the nucleotide-dependent conformational changes in the helicase. In further embodiments, each type of paired nucleotides produces a different conformational change in the helicase enzyme. In yet further embodiments, the different conformational changes may be structurally or temporally distinct or have different current blockage levels.

In some embodiments, the processive nucleic acid processing enzyme may be localized to the top opening or the bottom opening of the transmembrane pore. In other embodiments, the processive nucleic acid processing enzyme is localized to the transmembrane pore by covalent linkage to a nanopore-threading tether. In further embodiments, the threading tether includes polyethylene glycol (PEG) repeats that may be sufficient in length to span the transmembrane pore channel and may further include at least one current modulating substituent disposed within the PEG repeats. In other embodiments, the threading tether further includes a molecular anchor disposed at the opening of the transmembrane pore opposite the processive nucleic acid processing enzyme, which secures the tether in place within the pore. In yet further embodiments, the molecular anchor may be a double stranded oligonucleotide or a biotin-streptavidin conjugate. In other embodiments, the threading tether may be attached to a stationary domain or a mobile domain of the processive nucleic acid processing enzyme. In other embodiments, the processive nucleic acid processing enzyme is covalently attached to the transmembrane pore by at least one linker that may restrict substantial movement of the enzyme relative to the pore. In other embodiments, the processive nucleic acid processing enzyme is localized to the transmembrane pore by direct covalent linkage between a mobile domain in the enzyme and a position that blocks current flow in the transmembrane pore. In yet other embodiments, the processive nucleic acid processing enzyme and the transmembrane pore are expressed as a fusion protein. In yet another embodiment, the processive nucleic acid processing enzyme is localized within the transmembrane pore.

In some embodiments, the amino acid sequence of the processive nucleic acid processing enzyme may be genetically altered to modify the charge of the enzyme at the transmembrane pore interface or to optimize enzymatic activity in high salt buffers. In other embodiments, the transmembrane pore includes at least one current modulating substituent disposed in the interior of the pore.

In some embodiments, voltage drop across the transmembrane pore that induces ion current through the pore may be AC or DC.

In another embodiment, the nucleic acid remains external to the pore during processing by the processive nucleic acid processing enzyme.

In another aspect, the invention provides constructs including an ion conductive pore and a processive nucleic acid processing enzyme, in which the ion conductive pore has a top opening and a bottom opening with the enzyme localized proximal to one of the openings that undergoes conformational changes in response to processing of a nucleic acid external to the pore, and in which the conformational changes modulate current flow through the pore. In some embodiments, the ion conductive pore is a protein that may be αHL, MspA, or OmpG. In other embodiments, the processive nucleic acid processing enzyme is a DNA polymerase that may be Klenow fragment, Phi29, or DPO4. In yet other embodiments, the processive nucleic acid processing enzyme may be an exonuclease or a helicase. In other embodiments, the processive nucleic acid processing enzyme is localized to the ion conductive pore by covalent linkage to a threading tether that may include PEG repeats and may further be of a length sufficient to span the pore. In yet other embodiments, the tether may also include at least one current modulating substituent within the PEG repeats. In yet further embodiments, the threading tether may also include a molecular anchor at the end of the pore opposite that of the enzyme, which secures the tether in place within the pore and may be a double-stranded oligonucleotide or a biotin-streptavidin conjugate. In other embodiments, the threading tether may be attached to a stationary domain or a mobile domain of the enzyme. In yet other embodiments, the enzyme may be covalently attached to the pore by at least one linker that may restrict substantial movement of the enzyme relative to the pore. In other embodiments, the processive nucleic acid processing enzyme may be localized to the ion conductive pore by direct covalent linkage between a mobile domain in the enzyme and a position that blocks current flow in the pore. In another embodiment, the enzyme and the pore may be expressed as a non-natural fusion protein. In yet another embodiment, the enzyme may be localized within the pore. In other embodiments, the amino acid sequence of the processive nucleic acid processing enzyme may be genetically altered to modify the charge of the enzyme at the ion conductive pore interface or to optimize activity in high salt buffers.

In another aspect, the invention provides a system for determining the nucleotide sequence of a polynucleotide in a sample including: i) a cis chamber and a trans chamber, where the cis chamber and the trans chamber are separated by a membrane and where the cis and trans chambers include an electrically conductive mixture; ii) a construct according to any of the constructs described above assimilated with the membrane to provide a transmembrane pore and a processive nucleic acid processing enzyme, where the enzyme undergoes conformational changes in response to processing of the polynucleotide; iii) a reaction mixture in contact with the processive nucleic acid processing enzyme including reagents required for nucleic acid processing by the enzyme; iv) drive electrodes in contact with the electrically conductive reaction mixture on either side of the membrane for producing a voltage drop across the transmembrane pore; v) one or more measurement electrodes connected to electronic measurement equipment for measuring ion current through the transmembrane pore; and v) a computer to translate the ion current measurement into nucleic acid sequence information

In another aspect, the invention provides a method of assembling a molecular sensor complex including the steps of providing a transmembrane pore embedded in a membrane; delivering a processive nucleic acid processing enzyme-tether conjugate to a first side of the membrane, wherein the tether comprises a pore spanning segment, a first oligonucleotide segment and a tail segment of substantial negative charge; applying a voltage bias to the first side of the membrane sufficient to localize the conjugate to the transmembrane pore; and delivering a second oligonucleotide complementary to the first oligonucleotide segment to a second side of the membrane, wherein the second oligonucleotide hybridizes to the first oligonucleotide segment and secures the processive nucleic acid processing enzyme to the pore. In some embodiments, the processive nucleic acid processing enzyme is a DNA polymerase. In yet other embodiments, the DNA polymerase is the Klenow fragment of DNA polymerase I. In further embodiments, the Klenow fragment is a variant with amino acid substitutions C907S and L790C or C907S and S428C. In other embodiments, the transmembrane pore is αHL. In yet other embodiments, the pore spanning segment of the tether includes polyethylene glycol repeats and the tail segment of the tether includes phosphoramidite repeats.

These and other aspects of the invention will be apparent upon reference to the attached drawings and following detailed description. To this end, various references are set forth herein which describe in more detail certain procedures, compounds and/or compositions, and are hereby incorporated by reference in their entirety.

### BRIEF DESCRIPTION OF THE SEVERAL VIEWS OF THE DRAWINGS

In the figures, the sizes and relative positions of elements are not necessarily drawn to scale and some of these elements are arbitrarily enlarged and positioned to improve figure legibility. Further, the particular shapes of the elements as drawn are not intended to convey any information regarding the actual shape of the particular elements, and have been solely selected for ease of recognition in the figures.

FIGS. 1A and 1B show cartoons illustrating a method of the invention for nucleic acid sequencing using a generalized molecular sensor complex, which transduces nucleic acid processing events into electric signals. A nucleic acid processing enzyme localized to a nanopore embedded in a membrane undergoes a conformational change upon binding and processing a single nucleotide substrate. The conformational change is unique for each of the four individual nucleotides and results in a characteristic change in current through the nanopore that can be used to identify each nucleotide.

FIG. 2 is a flow chart illustrating an embodiment of a method of the invention for nucleic acid sequencing using a molecular sensor complex.

FIG. 3A shows a generic cartoon of one embodiment of a nanopore of the invention.

FIG. 3B shows a cartoon of one embodiment of a nanopore of the invention, here depicted as αHL.

FIG. 3C shows a cartoon of another embodiment of a nanopore of the invention, here depicted as OmpG.

FIG. 3D shows a cartoon of another embodiment of a nanopore of the invention, here depicted as MspA.

FIG. 4A shows the crystal structure of an exemplary DNA polymerase, illustrating relevant structural domains.

FIGS. 4B and 4C show cartoons illustrating an exemplary molecular sensor complex of the invention, composed of a DNA polymerase and a αHL nanopore. Here, the DNA polymerase is localized to the nanopore by a molecular tether, which is held in place, in turn, by a molecular anchor. The DNA polymerase undergoes a conformational change upon binding and incorporating a single nucleotide into a growing DNA strand. The conformational change is unique for each of the four individual nucleotides and results in a characteristic change in current that can be used to identify each nucleotide.

FIG. 5A shows one embodiment of the invention in which a nucleic acid processing enzyme is localized to a nanopore by a molecular tether and anchor.

FIG. 5B shows another embodiment of the invention in which a nucleic acid processing enzyme is localized to a nanopore by a molecular tether and anchor and a covalent linkage.

FIGS. 6A and 6B show another embodiment of the invention in which a nucleic acid processing enzyme is localized to a blocking position in a nanopore by a covalent linkage. A single nucleic acid processing event induces a conformational change in the enzyme, which opens the pore to current flow.

FIG. 7 shows another embodiment of the invention in which a nucleic acid processing enzyme is localized within the channel of a nanopore by a covalent linkage.

FIG. 8 shows another embodiment of the invention in which a nucleic acid processing enzyme and a nanopore are produced as a fusion protein.

FIG. 9 shows conjugation of the Klenow fragment (KF) DNA polymerase to two alternative molecular tethers.

FIG. 10A shows a signature electrical trace of an open nanopore and the nanopore partially occluded by a molecular tether.

FIG. 10B shows a signature electrical trace of an open nanopore and the nanopore occluded by a DNA polymerase conjugated to a molecular tether.

FIG. 11A shows conjugation of a variant Klenow fragment (KF) DNA polymerase with a repositioned conjugation site to different molecular tethers.

FIG. 11B shows DNA extension activity of wildtype and variant KF polymerases.

FIG. 12A shows optimization of DNA polymerase activity in high-salt buffers with different additives.

FIG. 12B shows optimization of DNA polymerase activity in high-salt buffers with different additives.

### DETAILED DESCRIPTION

### Definitions

The term “conformational change,” as used herein, when used in reference to an enzyme, means at least one change in the structure of the enzyme, a change in the shape of the enzyme or a change in the arrangement of parts of the enzyme or a shift or a change in charge distribution. The enzyme can be, for example, a polymerase, exonuclease, helicase, or other processive nucleic acid processing enzyme such as those set forth herein below. The parts of the enzyme can be, for example, atoms that change relative location due to rotation about one or more chemical bonds occurring in the molecular structure between the atoms. The parts can also be regions of secondary, tertiary or quaternary structure. The parts of the enzyme can further be domains of a macromolecule, such as those commonly known in the relevant art. For example, polymerases include domains referred to as the finger, palm and thumb domains.

The term “transmembrane pore,” as used herein, generally refers any structure that conducts current from one reservoir to another; transmembrane pores may also be referred to herein as “ion conductive pores” or, alternatively, “electroconductive pores”. A transmembrane pore may be a pore, channel or passage formed or otherwise provided in a membrane that permits hydrated ions to flow from one side of a membrane to the other side of the membrane. A transmembrane pore can be defined by a molecule in a membrane, or other suitable substrate. A transmembrane pore may be defined by a multiple of smaller pores within a defined boundary acting collectively like a single pore. Some transmembrane pores are protein nanopores and may be a single polypeptide or a collection of polypeptides made up of several repeating subunits. Alpha hemolysin (αHL), OmpG, and MspA are examples of suitable protein nanopores of the invention. A transmembrane pore may also be defined by a solid-state nanopore. A transmembrane pore may have a characteristic width or diameter on the order of 0.1 nanometers (nm) to about 1000 nm. A transmembrane pore may be disposed adjacent or in proximity to a sensing circuit, such as, for example, a complementary metal-oxide semiconductor (CMOS) or field effect transistor (FET) circuit

A “membrane” as used herein is a thin film that separates two compartments or reservoirs and prevents the free diffusion of ions and other molecules between these. Suitable membranes are amphiphilic layers formed of amphiphilic molecules, i.e., molecules possessing both hydrophilic and lipophilic properties. Such amphiphilic molecules may be either naturally occurring, such as phospholipids, or synthetic. Examples of synthetic amphiphilic molecules include such molecules as poly(n-butyl methacrylate-phosphorylcholine), poly(ester amide)-phosphorylcholine, polylactide-phosphorylcholine, polyethylene glycol-poly(caprolactone)-di- or tri-blocks, polyethylene glycol-polylactide di- or tri-blocks and polyethylene glycol-poly(lactide-glycolide) di- or tri-blocks. Preferably, the amphiphilic layer is a lipid bilayer. Lipids bilayers are models of cell membranes and have been widely used for experimental purposes. A membrane can also be a solid-state membrane, i.e., a layer prepared from solid-state materials in which one or more aperture is formed. The membrane may be a layer, such as a coating or film on a supporting substrate, or it may be a free-standing element. Examples of materials used for thin film solid state membranes include silicon nitride, aluminum oxide, titanium oxide, and silicon oxide.

“Nucleobase” is a heterocyclic base such as adenine, guanine, cytosine, thymine, uracil, inosine, xanthine, hypoxanthine, or a heterocyclic derivative, analog, or tautomer thereof. A nucleobase can be naturally occurring or synthetic. Non-limiting examples of nucleobases are adenine, guanine, thymine, cytosine, uracil, xanthine, hypoxanthine, 8-azapurine, purines substituted at the 8 position with methyl or bromine, 9-oxo-N-6-methyladenine, 2-aminoadenine, 7-deazaxanthine, 7-deazaguanine, 7-deaza-adenine, N4-ethanocytosine, 2,6-diaminopurine, N6-ethano-2,6-diaminopurine, 5-methylcytosine, 5-(C3-C6)-alkynylcytosine, 5-fluorouracil, 5-bromouracil, thiouracil, pseudoisocytosine, 2-hydroxy-5-methyl-4-triazolopyridine, isocytosine, isoguanine, inosine, 7,8-dimethylalloxazine, 6-dihydrothymine, 5,6-dihydrouracil, 4-methyl-indole, ethenoadenine and the non-naturally occurring nucleobases described in U.S. Pat. Nos. 5,432,272 and 6,150,510 and PCT applications WO 92/002258, WO 93/10820, WO 94/22892, and WO 94/24144, and Fasman (“Practical Handbook of Biochemistry and Molecular Biology”, pp. 385-394, 1989, CRC Press, Boca Raton, La.), all herein incorporated by reference in their entireties.

“Nucleobase residue” includes nucleotides, nucleosides, fragments thereof, and related molecules having the property of binding to a complementary nucleotide. Deoxynucleotides and ribonucleotides, and their various analogs, are contemplated within the scope of this definition. Nucleobase residues may be members of oligomers and probes. “Nucleobase” and “nucleobase residue” may be used interchangeably herein and are generally synonymous unless context dictates otherwise.

“Polynucleotides”, also called nucleic acids, are covalently linked series of nucleotides in which the 3′ position of the pentose of one nucleotide is joined by a phosphodiester group to the 5′ position of the next. DNA (deoxyribonucleic acid) and RNA (ribonucleic acid) are biologically occurring polynucleotides in which the nucleotide residues are linked in a specific sequence by phosphodiester linkages. As used herein, the terms “polynucleotide” or “oligonucleotide” encompass any polymer compound having a linear backbone of nucleotides. Oligonucleotides are generally shorter chained polynucleotides. Nucleic acid are generally referred to as “target nucleic acid” if targeted for sequencing.

“Complementary” generally refers to specific nucleotide duplexing to form canonical Watson-Crick base pairs, as is understood by those skilled in the art. However, complementary as referred to herein also includes base-pairing of nucleotide analogs, which include, but are not limited to, 2′-deoxyinosine and 5-nitroindole-2′-deoxyriboside, which are capable of universal base-pairing with A, T, G or C nucleotides and locked nucleic acids, which enhance the thermal stability of duplexes. One skilled in the art will recognize that hybridization stringency is a determinant in the degree of match or mismatch in the duplex formed by hybridization.

“Nucleic acid” is a polynucleotide or an oligonucleotide. A nucleic acid molecule can be deoxyribonucleic acid (DNA), ribonucleic acid (RNA), or a combination of both. Nucleic acids are generally referred to as “target nucleic acids” or “target sequence” if targeted for sequencing. Nucleic acids can be mixtures or pools of molecules targeted for sequencing.

“Hybridize” shall mean the annealing of one single-stranded nucleic acid to another nucleic acid (such as primer) based on the well-understood principle of sequence complementarity. In an embodiment the other nucleic acid is a single-stranded nucleic acid. The propensity for hybridization between nucleic acids depends on the temperature and ionic strength of their milieu, the length of the nucleic acids and the degree of complementarity. The effect of these parameters on hybridization is described in, for example, Sambrook J, Fritsch E F, Maniatis T., Molecular cloning: a laboratory manual, Cold Spring Harbor Laboratory Press, New York (1989). As used herein, hybridization of a primer sequence, or of a DNA extension product, to another nucleic acid shall mean annealing sufficient such that the primer, or DNA extension product, respectively, is extendable by creation of a phosphodiester bond with an available nucleotide or nucleotide analogue capable of forming a phosphodiester bond, therewith.

“Primer” as used herein (a primer sequence) is a short, usually chemically synthesized oligonucleotide, of appropriate length, for example about 18-24 bases, sufficient to hybridize to a target DNA (e.g., a single stranded DNA) and permit the addition of a nucleotide residue thereto, or oligonucleotide or polynucleotide synthesis therefrom, under suitable conditions. In an embodiment the primer is a DNA primer, i.e., a primer consisting of, or largely consisting of, deoxyribonucleotide residues. The primers are designed to have a sequence which is the reverse complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product.

A “daughter strand” is produced by a template-directed process and is generally complementary to the target single-stranded nucleic acid from which it is synthesized.

“Tether” or “tether member” refers to a polymer or molecular construct having a generally linear dimension and with an end moiety at each of two opposing ends. A tether is attached to a molecular structure (e.g., a protein subunit or other substrate) with a linkage in at least one end moiety to form a tether construct.

“Moiety” is one of two or more parts into which something may be divided, such as, for example, the various parts of a tether, a molecule or a probe.

“Processive” refers to a process of coupling of substrates which is generally continuous and proceeds with directionality. While not bound by theory, polymerases, exonucleases, and helicases, for example, exhibit processive behavior if nucleic acid substrates are processed incrementally without interruption. The steps of polymerization, degradation, or strand unwinding are not seen as independent steps if the net effect is processive processing.

“Linker” is a molecule or moiety that joins two molecules or moieties, and provides spacing between the two molecules or moieties such that they are able to function in their intended manner. For example, a linker can comprise a diamine hydrocarbon chain that is covalently bound through a reactive group on one end to an oligonucleotide analog molecule and through a reactive group on another end to a solid support, such as, for example, a bead surface. Coupling of linkers to enzymes, pores, and tethers or substrate constructs of interest can be accomplished through the use of coupling reagents that are known in the art (see, e.g., Efimov et al., Nucleic Acids Res. 27: 4416-4426, 1999). Methods of derivatizing and coupling organic molecules are well known in the arts of organic and bioorganic chemistry. A linker may also be cleavable or reversible.

The articles “a”, “an” and “the” are non-limiting. For example, “the method” includes the broadest definition of the meaning of the phrase, which can be more than one method.

All publications, patents, and patent applications cited herein, whether supra or infra, and hereby incorporated by reference in their entirety.

Nucleic Acid Sequencing with Molecular Sensor Complexes

The invention is generally directed to methods, constructs, and systems for sequencing nucleic acids. In particular, the invention is directed to real time (i.e., as it occurs), single molecule (i.e., single nucleotide processing) sequencing of nucleic acids using current-conducting transmembrane pores and processive nucleic acid processing enzymes. By exploiting the sensitivity of electronic detection, the present invention offers considerable advantages over real time single molecule sequencing systems relying on optical detection methods, which generate less reliable and lower signals with increased noise. Furthermore, the electronic detection methods of the present invention are not dependent upon translocation of a nucleic acid substrate through a transmembrane pore, thus overcoming the nucleotide resolution limitations hindering current nanopore-based electronic sequencing systems. The sequencing methods of the present invention are also advantageous in that they can interrogate natural enzyme-substrate interactions, thus avoiding complications potentially introduced by use of non-natural, synthetic substrates or enzymes.

As further discussed below, an enzyme that physically obstructs an opening, or aperture, of a transmembrane pore will likewise disrupt the flow of current through the pore. Moreover, an enzyme with the ability to assume different blocking conformations will differentially disrupt current flow and therefore generate electronic signals specific for unique enzymatic conformations. By recording electronic signals through a pore over time as an enzyme moves through various conformations, information about enzyme-substrates interactions can be indirectly obtained. Such macromolecular constructs comprising a conformationally flexible enzyme localized to a transmembrane pore are herein referred to as molecular “sensor complexes”.

In particular embodiments, a sequence of nucleotides of a target nucleic acid is determined based on the succession of conformational changes an enzyme transitions through as it interacts with the nucleic acid. Such enzymes, herein referred to as processive nucleic acid processing enzymes, interact sequentially with the nucleotide subunits of a nucleic acid in order to carry out a series of reactions on the nucleic acid. Distinguishing the conformational changes that occur for each type of nucleotide the enzyme interacts with and determining the sequence of those changes can be used to determine the sequence of the nucleic acid. For example, a DNA polymerase can use a first nucleic acid strand as a template to sequentially build a second, complementary nucleic acid strand by sequential addition of nucleotides to the second strand. The polymerase undergoes conformational changes with each nucleotide addition. As set forth in further detail herein, the conformational changes that occur for each type of nucleotide that is added can be distinguished and the sequence of those changes can be detected to determine the sequence of either or both of the nucleic acid strands. In another example, an exonuclease can sequentially remove nucleotides from a nucleic acid. Conformational changes that occur for each type of nucleotide that is removed can be distinguished and the sequence of those changes can be detected to determine the sequence of the nucleic acid. In yet another example, a helicase can sequentially separate paired nucleotides in a doubled stranded nucleic acid. Conformational changes that occur for each type of nucleotide that is separated can be distinguished and the sequence of those changes can be detected to determine the sequence of the nucleic acid. In addition to these exemplary processive nucleic acid processing enzymes, any enzyme that processively interacts with individual nucleotide subunits of a target nucleic acid while undergoing nucleotide-specific conformational changes can be suitable for the practice of the present invention.

In one embodiment of the present invention, the conformational movements of processive nucleic acid processing enzymes are transduced into electric signals by a transmembrane pore. Current flowing through a pore is modulated, depending on the particular conformation of an enzyme localized to a pore opening. These electronic signals provide a means for identifying individual nucleotides associated with the processing enzyme. As each different nucleotide induces a distinct enzymatic conformation, the identity of the nucleotide bound by the enzyme can be determined by observing current modulations through the pore over time. For example, a nucleotide which is processed may spend more time associated with the enzyme than nucleotides which are not processed, allowing for identification, or “calling” of bases based on current modulation amplitude, duration, or other characteristics.

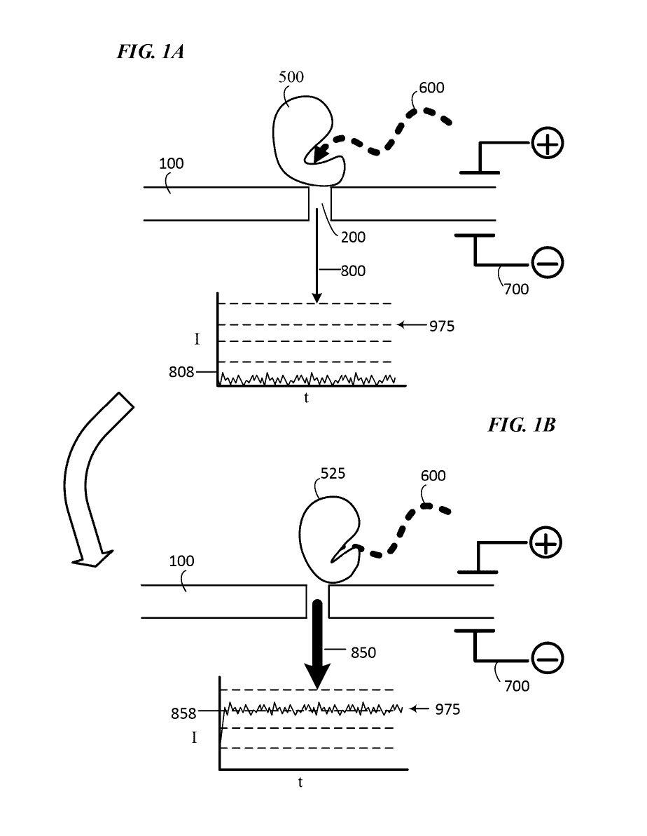

FIGS. 1A and 1B are cartoons representing a generalized molecular sensor complex of the present invention. For clarity of discussion, features illustrated in the figures are not shown to scale. The sensor complex is comprised of processive nucleic acid processing enzyme 500 localized to transmembrane pore 200 in membrane 100. In this configuration, the enzyme physically obstructs the opening to the pore. The enzyme may be localized to the top opening, as depicted here; alternatively, the enzyme may be localized to the bottom opening of the pore or within the pore itself. The degree to which the enzyme physically obstructs the pore may be partial or complete. The enzyme is complexed with target nucleic acid 600, which is processed by the enzyme in a processive manner and with a directionality here indicated by the arrow head. In this embodiment, the target nucleic acid remains external to the pore and does not physically obstruct the pore opening. The enzyme may be localized to the pore through non-covalent interactions, such as ionic or hydrophobic interactions, or preferably by covalent attachment to a tether element and/or to the pore itself as described in further detail below. Alternatively, a non-natural fusion protein is synthesized with two functional parts; one that performs the enzymatic function and one that functions as the transmembrane pore. The assembly of the enzyme and the transmembrane pore may be referred to as a “construct”, which is a stable complex of more than one polypeptide that do not normally function together in nature. The membrane separates two reservoirs that contain a conductive solution with high concentrations of electrolyte, such as 1M KCl. Electrodes (e.g., Ag/AgCl) are placed in each reservoir with an applied potential between them to form voltage drop 700 across the membrane, enabling an ion current flow 800 through the pore to be measured in an external circuit that completes the circuit between the electrodes. In other embodiments, the applied potential may drive the current in the opposite direction and/or as an alternating current since there is no additional requirement to drive the nucleic acid through the pore as seen in other nanopore technologies. In this exemplary illustration, the enzyme is depicted in a first conformation 500 that is induced by an interaction with a first subunit of the target nucleic acid. While in this first conformation, the enzyme substantially blocks current flow 800 through the membrane, such that recorded current level 808 is relatively low.

The transition of the sensor complex configuration to that illustrated in FIG. 1B corresponds to a single nucleic acid processing event, or nucleotide-dependent activity, executed by the enzyme. In this second configuration, the enzyme assumes a second conformation 525, in which the physical block to the pore opening is substantially reduced, resulting in an increase in current flow 850 through the pore. To identify the specific nucleotide processed by the enzyme during the transition, the recorded current 858 is correlated to current modulation characteristic 975. The current modulation characteristic depicted in this embodiment reflects an electronic signal of a specific amplitude; however the current modulation characteristic may, alternatively, be temporal. Each nucleotide subunit of the target nucleic acid has a specific current modulation characteristic, which allows for base calling as the molecular sensor complex processes the target nucleic acid. Alternatively, each nucleotide type is presented to the sensor in a cyclic sequential manner and only the current modulation of an incorporation event (of any type) need be recorded since the nucleotide type is determined by the cycle timing.

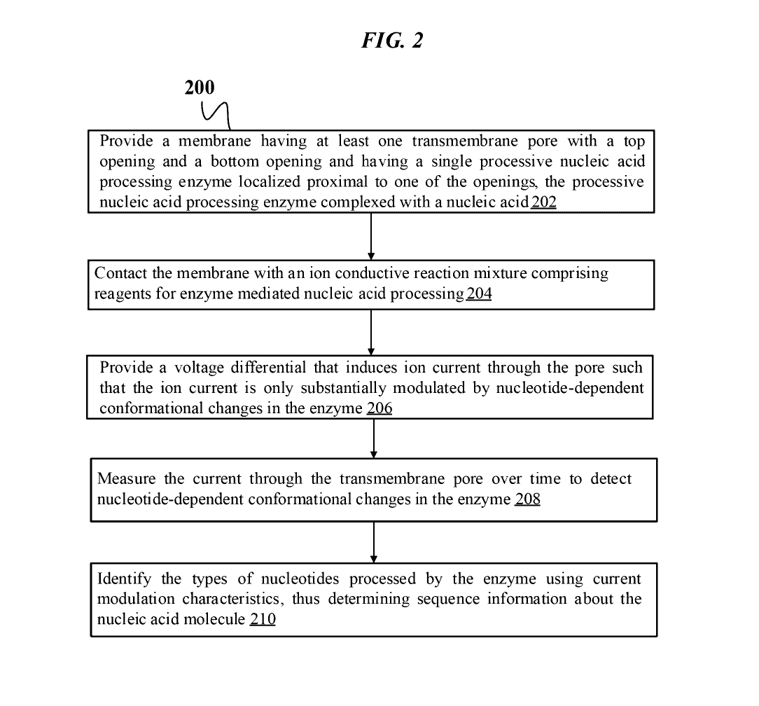

A generalized method for determining sequence information about a nucleic acid molecule is depicted in the flow chart of FIG. 2. Method 200 includes the steps of providing a membrane having at least one transmembrane pore with a top opening and a bottom opening, and having a single processive nucleic acid processing enzyme localized proximal to one of the openings, the processive nucleic acid processing enzyme complexed with a nucleic acid 202 external to the pore; contacting the membrane and the processive nucleic acid processing enzyme with an ion conductive reaction mixture comprising reagents for nucleic acid processing by the enzyme 204; providing a voltage differential that induces ion current through the pore such that the ion current is only substantially modulated by nucleotide-dependent conformational changes in the enzyme 206; measuring the current through the transmembrane pore over time to detect the nucleotide-dependent conformational changes in the processive nucleic acid processing enzyme 208; and identifying the types of nucleotides processed by the processive nucleic acid processing enzyme using current modulation characteristics, thus determining sequence information about the nucleic acid molecule 210. As used herein, “nucleotide-dependent conformational changes” may be induced by any molecular event that occurs as an enzyme processes, or carries out a chemical reaction, on a single monomeric unit of a target nucleic acid substrate. Examples of molecular events (i.e., nucleotide processing events) include, but are not limited to, template-dependent incorporation of a single nucleobase into a growing nucleic acid strand as may occur with a polymerase, removal of a single nucleobase from a single or double-stranded nucleic acid as may occur with an exonuclease, or separation of a single pair of nucleobases in a double stranded nucleic acid as may occur with a helicase.

### Transmembrane Pores

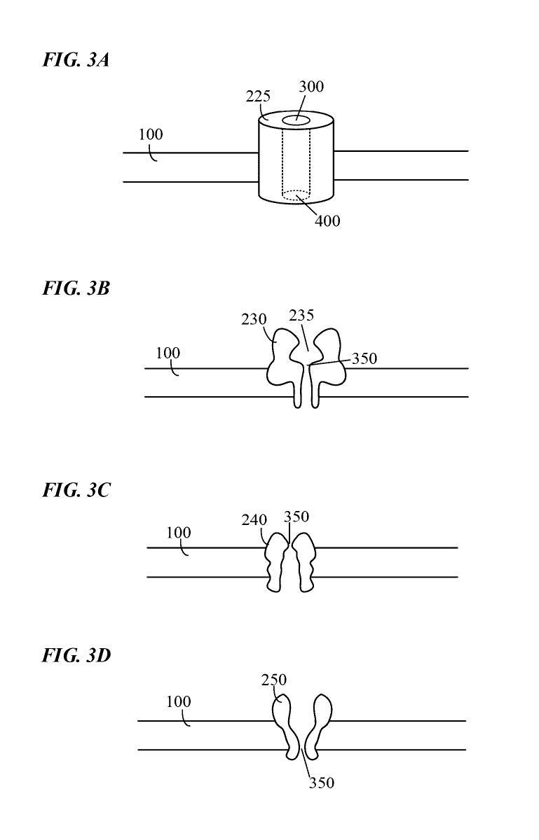

FIGS. 3A-3D illustrate various alternative embodiments of the transmembrane pore of the present invention. As shown in FIG. 3A, the pore can be defined by a molecule 225 with top opening 300 and bottom opening 400 in membrane 100. In some embodiments, the top and/or bottom openings may be only transiently formed in the sensor complex. The molecule may be a protein nanopore, a solid-state nanopore, or a hybrid of a solid-state nanopore and a protein nanopore. Protein nanopores have the advantage that as biomolecule, they self-assemble and can be identical to one another. In addition, it is possible to genetically engineer them to confer desired attributes or to create a fusion protein (e.g., fusion with a processive nucleic acid processing enzyme). Additional embodiments include transmembrane pores formed in lipid bilayers that are unnatural synthetic biological nanopores such as modified DNA oragami pores (Burns, J. R., Stulz, E., & Howorka, S. (2013). Self-Assembled DNA Nanopores That Span Lipid Bilayers. Nano Letters, 13(6), 2351-2356.), metal-organic channels, pi-stacks, crown ethers or other macrocycles (Sakai, N., & Matile, S. (2013). Synthetic Ion Channels. Langmuir, 29(29), 9031-9040). On the other hand, solid state nanopore have the advantage that they are more robust and stable compared to a protein embedded in a lipid membrane. Furthermore, solid state nanopores can in some cases be multiplexed and batch fabricated in an efficient and cost-effective manner. Finally, they might be combined with micro-electronic fabrication technology. Solid state nanopores are pores that are formed in a membrane fabricated using solid state processes. A common solid state membrane is a silicon nitride thin film formed on a silicon wafer using a chemical vapor deposition process and where the silicon is subsequently etched away. The pore may be formed by drilling with a transmission electron beam microscope and its size can be chosen to optimize the sensor performance. In some cases, small pores from about 0.1 nanometer to about 5 nanometers in diameter are used. In other applications, pores from about 2 nanometers to about 10 nanometers are used. In yet other embodiments, pores of up to 1000 nanometers are used. In some embodiments protein nanopores may be supported or embedded in a solid state pore and thus have a solid state membrane.

In one embodiment of the present invention, the transmembrane pore is a protein nanopore. In some cases, as depicted in FIG. 3B, the nanopore is formed by α-hemolysin (αHL) protein 230. αHL is a monomeric polypeptide which self-assembles, e.g., in a lipid bilayer membrane, to form a heptameric pore, with a 2.6 nm-diameter vestibule 235 and 1.5 nm-diameter limiting aperture (the narrowest point of the pore) 350. The limiting aperture of the αHL nanopore allows linear molecules, with dimensions on the order of that of single-stranded DNA, to pass through, or “translocate”; however molecules with a diameter larger the ˜2.0 nm, such as double-stranded DNA, are precluded from translocation. In other cases, as depicted in FIG. 3C, the nanopore is formed by E. coli outer membrane protein G (OmpG) 240. Limiting aperture 350 of OmpG is found at the top opening, which is 0.8 nm diameter, while the diameter of the bottom opening is 1.4 nm. OmpG is composed of β-strands connected by seven flexible loops on the top side. OmpG spontaneously gates during an applied potential, due to one of the flexible loops, which flops in and out of the pore, intermittently blocking the current. In some embodiments, modulations of this gating pattern may be used as current modulation characteristics. In other cases, as depicted in FIG. 3D, the nanopore is formed by Mycobacterium smegmatis porin (MspA) protein 250. Limiting aperture 350 of MspA is found at the bottom of the funnel shaped protein with a diameter of 1.2 nm. In an aqueous ionic salt solution, e.g., 1M KCl, when an appropriate voltage is applied across the membrane, the pore formed by any of these embodiments conducts a sufficiently strong and steady ionic current for the practice of the present invention.

In certain embodiments, the nanopore protein may be modified to optimize signal transduction by the molecular sensor complex. Modifications may include one or more alterations of the amino acid residues at the surface of the pore lumen. Such modifications may alter interactions with any of the components of the sensor complex, described in more detail below. Methods of protein engineering are well known in the art and discussed further herein.

In some instances, a transmembrane pore is inserted into a lipid bilayer membrane (e.g., by electroporation). The transmembrane pore can be inserted spontaneously, during membrane formation, or by a stimulus signal such as an electrical stimulus, a pressure stimulus, a liquid flow stimulus, a gas bubble stimulus, sonication, sound, vibration, or any combination thereof. In other instances, a transmembrane pore is drilled into a solid state thin film by a transmission electron microscope, or a helium ion microscope, or by etching through holes in a resist that are defined by an electron beam.

As disclosed herein, a processive nucleic acid processing enzyme is localized, located in proximity to, or attached to the transmembrane pore before or after the pore is incorporated into the membrane. In some instances, the transmembrane pore and enzyme are a non-natural fusion protein (i.e., expressed as a single polypeptide chain). The processive nucleic acid processing enzyme can be localized, located in proximity to, or attached to the transmembrane pore in any suitable way. In some cases, the enzyme is covalently linked to one of the protein monomers in a multimonomer nanopore protein. For example, a linked αHL (heptamer) nanopore, can be assembled by mixing its constituent monomers, in the ratio of one enzyme linked monomer to 6 unmodified monomers in the presence of liposomes to help catalyze the assembly. Fully assembled nanopores can then be purified and size-selected for those that have only a single linked enzyme. These assembled nanopores can then be inserted into the membrane. Other means of attaching or localizing an enzyme to a pore are described in further detail below.

### Processive Nucleic Acid Processing Enzymes

The constructs and methods of the current invention provide improved sequencing accuracy by concurrently observing enzyme conformation and nucleotide processing (i.e., nucleotide-dependent activity). The processive nucleic acid processing enzyme undergoes a series of conformational changes during the process of, e.g., polymerizing a daughter strand off a template nucleic acid, removing nucleotides from a double stranded or single stranded nucleic acid, or separating or unwinding the two strands of a double stranded nucleic acid. During these conformational changes, various regions or domains of the enzyme can move relative to one another. It has been recently shown that such conformational changes can be observed in real time, even at the single-molecule level (see, e.g., Gill et al. Biochem. Soc. Trans., 39: 595, 2011). By observing conformational changes in the enzyme in real time while the enzyme is processing nucleotides, it is possible to distinguish true events from other events which might otherwise be mistaken as true events.

### Conformational Changes of DNA Polymerase

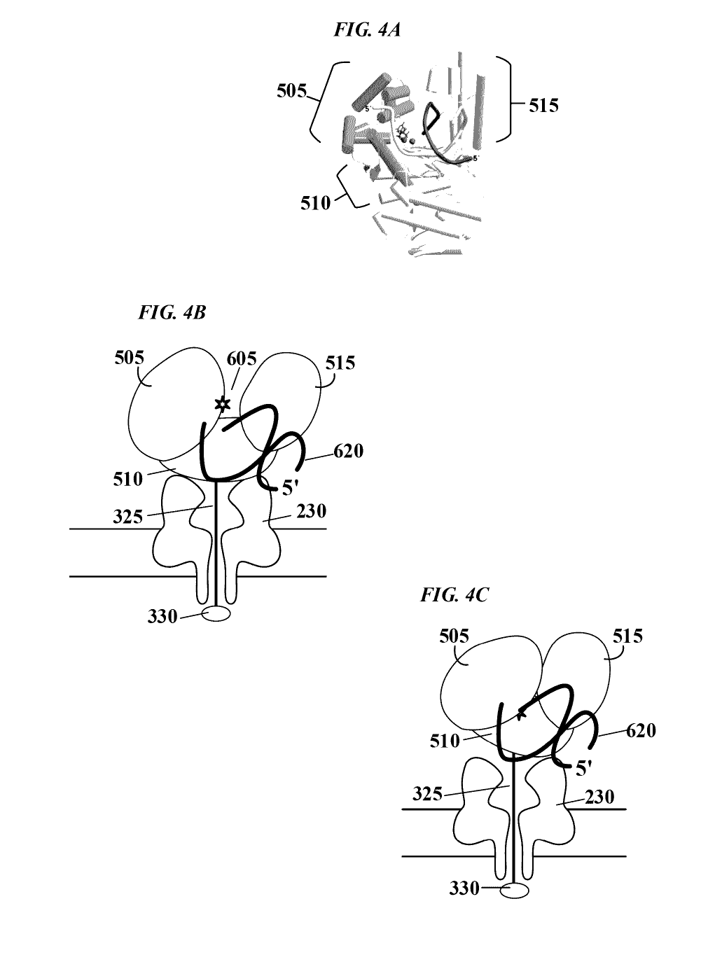

DNA polymerases are by their very nature small machines. Polymerases are made up of domains, which, like parts of a machine, can move relative to one another during the polymerase reaction. The major domains common to DNA polymerases are illustrated in FIG. 4A. The structure of a DNA polymerase is analogous to a right hand with “finger” domain 505, and “palm” domain 510 and “thumb” domain 515. A function of the palm domain is catalysis of the phosphoryl transfer reaction whereas that of the finger domain includes important interactions with the incoming nucleoside triphosphate as well as the template base to which it is paired. The thumb domain, on the other hand, may play a role in positioning the duplex DNA and in processivity and translocation (see, e.g., Joyce et al. Biochemistry, 43: 14324, 2004).

Polymerases undergo conformational changes in the course of synthesizing a nucleic acid polymer. For example, polymerases undergo a conformational change from an open conformation to a closed conformation upon binding of a nucleotide. A polymerase that is bound to a nucleic acid template and growing primer with no free nucleotide present is in what is referred to in the art as an “open” conformation. When this polymerase complexes with a nucleotide that is the complement to the template base in the next extension position the polymerase reconfigures into what is referred to in the art as a “closed” conformation. At a more detailed structural level, the transition from the open to closed conformation is characterized by relative movement within the polymerase resulting in the “thumb” domain and “fingers” domain being closer to each other. In the open conformation the thumb domain is further from the fingers domain, akin to the opening and closing of the palm of a hand. In various polymerases, the distance between the tip of the finger and the thumb can change up to 10 angstroms between the “open” and “closed” conformations. The distance between the tip of the finger and the rest of the protein domains can also change up to 10 Angstroms. It will be understood that this change will be exploited in a method set forth herein. Furthermore, other changes can be exploited including those that are less than 10, 8, 6, 4, or 2 Angstroms so long and including those that are greater than 10 Angstroms.

DNA polymerases undergo several kinetic transitions in the course of adding a nucleotide to a growing nucleic acid strand. Distinguishable transitions include, for example, the binding of a nucleotide to the polymerase-nucleic acid complex to form an open polymerase-nucleic acid-nucleotide ternary complex, the transition of the polymerase in the open polymerase-nucleic acid-nucleotide ternary complex to the closed polymerase′-nucleic acid-nucleotide ternary complex, catalytic bond formation between the nucleotide and nucleic acid in the closed polymerase′-nucleic acid-nucleotide ternary complex to form a closed polymerase′-extended nucleic acid-pyrophosphate complex, transition of the closed polymerase′-extended nucleic acid-pyrophosphate complex to an open polymerase-extended nucleic acid-pyrophosphate complex, release of pyrophosphate from the open polymerase-extended nucleic acid-pyrophosphate complex to form an open polymerase-extended nucleic acid complex, and eventual (i.e., optionally after several repetitions of nucleotide binding incorporation) release of the extended nucleic acid from the open polymerase-extended nucleic acid complex to form the uncomplexed polymerase. One or more of the transitions that a polymerase undergoes when adding a nucleotide to a nucleic acid can be detected using a molecular sensor complex as described herein.

The generalized cartoons in FIGS. 4B and 4C illustrate the major structural domains of a DNA polymerase and their relative locations in “open” and “closed” conformations. For clarity, the polymerase structure in the figures is reduced to elements that illustrate some relevant features of finger domain movements. The conformation of the polymerase in the open structure is shown in FIG. 4B. Upon binding of incoming nucleotide 605, finger domain 505 in the binary complex of polymerase and DNA moves closer to thumb domain 515 as indicated in FIG. 4C.

In particular embodiments, with continued reference to FIGS. 4B and 4C, the conformational movement of DNA polymerase in a molecular sensor complex (herein referred to as a “polymerase sensor complex”) can be used to distinguish the species of nucleotide 605 that is added to primed nucleic acid template 620. In this exemplary embodiment, DNA polymerase is localized to αHL nanopore 230 by covalent attachment, or conjugation, to tether 325. The enzyme/tether conjugate is held in place on the cis side of the membrane by anchor 330, which is restricted to the trans side of the membrane (i.e., the distal side relative to the enzyme). Other means of localizing the enzyme to the pore are contemplated by the present invention and further described below. In this embodiment, the tether is conjugated to palm domain 510 of the polymerase enzyme. In other embodiments, the tether may be conjugated to mobile finger domain 505 or thumb domain 515. FIG. 4B depicts the polymerase in the “open” configuration in which it is bound to primed target nucleic acid 620, but not bound to incoming nucleotide 605. In this “open” configuration, the polymerase substantially occludes the top opening of the nanopore and consequently will substantially restrict the flow of ion current through the pore during an applied potential, as discussed with reference to FIGS. 1A and 1B.

FIG. 4C depicts the polymerase in a second, ™, “closed” configuration, which is induced, e.g., by binding of incoming nucleotide 605 to form a correct base pair with the template nucleic acid. In this second configuration, the degree to which the enzyme physically occludes the pore is reduced, and consequently the flow of current through the pore will increase. Such modulation of current flow generates an electronic signal specific for nucleotide species 605. Electronic signals measured over time as the polymerase sensor complex synthesizes a daughter strand provides sequence information in real time based on the current modulation characteristics of each of the four individual nucleotides.

The above illustrations are for explanatory purposes only. For the methods and constructs of the present invention, it is not required that there exist distinct states in order for a measurement of conformational change to occur. What is required is that the signal that is sensitive to enzyme conformation changes reproducibly during the polymerase reaction. For example, as one portion of the enzyme moves relative to another portion of the enzyme during the polymerase reaction, one portion of the enzyme may sweep past another portion of the enzyme during one or more steps. Where this occurs, for instance, the flow of ion current through the pore is altered in a characteristic manner. Thus, in some cases of the invention there are two, three, four or more discrete states which can be identified that result in different signal levels. In some cases, the signal will result from transient signals generated as the enzyme moves, for example, from one state to another.

### Current Modulation Characteristics

Current modulation characteristics indicate base incorporation (or other individual nucleotide processing events for other enzymes) and can allow for base discrimination, or “base-calling”. In one embodiment, each of the four nucleotides induces a different polymerase conformation, as illustrated in FIG. 4C. The movement of the polymerase during the incorporation of a nucleotide will modulate the ion current through the pore in a characteristic and reproducible manner, generating a signature electric signal. In one embodiment, the current modulation shows a characteristic change in current amplitude that can be expressed as a ratio of I (altered current level) to Io (baseline). In another embodiment, the current modulation has a characteristic time duration of a single nucleotide's binding and incorporation by a polymerase that may be recorded as the shape of the measured current. In another embodiment, the average amplitude of the current modulation doesn't change, but rather the noise in the current modulation changes as a single nucleotide is bound and incorporated. In yet another embodiment, the current modulation system only indicates an incorporation event but does not discriminate the base type. In this embodiment, the sequence information about a nucleic acid is obtained by sequentially flooding the senor complex with one of four reaction mixtures containing one of the four nucleotides and detecting the presence or absence of an electric signal. Specifically, a nucleotide species that base-pairs with A can be added in a first reaction, a nucleotide species that base-pairs with C can be added in a second reaction, a nucleotide species that base-pairs with T can be added in a third reaction, and a nucleotide species that base-pairs with G can be added in a fourth reaction. The reactions are referred to as first, second, third and fourth merely to illustrate that the reactions are separate but this does not necessarily limit the order by which the species can added in a method set forth herein. Rather, nucleotide species that base-pair with A, C, T or G can be added in any order desired or appropriate for a particular embodiment of the methods. Any of a variety of detection techniques known in the art can be used including, but not limited to CMOS-based detection systems.

In some embodiments, the nucleotide being processed by the enzyme is modified such that the conformational change of the enzyme is larger or otherwise differentiated from the conformational changes induced by the other three nucleotides (which may or may not be modified). In these embodiments, the enzyme may have been modified by mutagenesis to perform better with modified nucleotides and produce enhanced signals. An example of highly modified bases are XNTPs that are polymerized in a template-dependent manner using, e.g., DPO4 polymerase variants that have been evolved to accept XNTPs as substrates.

This sensor technology performs single molecule measurements while sequentially advancing along a target nucleic acid to determine base identities in sequence in real time.

### DNA Polymerases

Polymerase enzymes that are suitable for the molecular sensor complexes and sequencing methods of the present invention may include any suitable polymerase enzyme capable of template directed nucleic acid synthesis. DNA polymerases are sometimes classified into six main groups based upon various phylogenetic relationships, e.g., with E. coli Pol I (class A), E. coli Pol II (class B), E. coli Pol III (class C), Euryarchaeotic Pol. II (class D), human Pol beta (class X), and E. coli UmuC/DinB and eukaryotic RAD30/xeroderma pigmentosum variant (class Y). For a review of recent nomenclature, see, e.g., Burgers et al. (2001) “Eukaryotic DNA polymerases: proposal for a revised nomenclature” J Biol. Chem. 276(47):43487-90. For a review of polymerases, see, e.g., Hubscher et al. (2002) “Eukaryotic DNA Polymerases” Annual Review of Biochemistry Vol. 71: 133-163; Alba (2001) “Protein Family Review: Replicative DNA Polymerases” Genome Biology 2(1): reviews 3002.1-3002.4; and Steitz (1999) “DNA polymerases: structural diversity and common mechanisms” J. Biol. Chem. 274:17395-17398. The sequences of literally hundreds of polymerases are publicly available, and the crystal structures for many of these have been determined, or can be inferred based upon similarity to solved crystal structures for homologous polymerases. For example, the crystal structure of DPO4, Klenow fragment, and Phi29, certain preferred enzymes to be used in a molecular sensor complex are available.

Polymerases can be characterized according to their processivity. A polymerase can have an average processivity that is at least about 50 nucleotides, 100 nucleotides, 1,000 nucleotides, 10,000 nucleotides, 100,000 nucleotides or more. Alternatively or additionally, the average processivity for a polymerase used as set forth herein can be, for example, at most 1 million nucleotides, 100,000 nucleotides, 10,000 nucleotides, 1,000 nucleotides, 100 nucleotides or 50 nucleotides. Polymerases can also be characterized according to their rate of processivity or nucleotide incorporation. For example, many native polymerases can incorporate nucleotides at a rate of at least 1,000 nucleotides per second. In some embodiments a slower rate may be desired. For example, an appropriate polymerase and reaction conditions can be used to achieve an average rate of at most 500 nucleotides per second, 100 nucleotides per second, 10 nucleotides per second, 1 nucleotide per second, 1 nucleotide per 10 seconds, 1 nucleotide per minute or slower. As set forth in further detail elsewhere herein, nucleotide analogs can be used that have slower or faster rates of incorporation than naturally occurring nucleotides. It will be understood that polymerases from any of a variety of sources can be modified to increase or decrease their average processivity or their average rate of processivity (e.g., average rate of nucleotide incorporation) or both. Accordingly, a desired reaction rate can be achieved using appropriate polymerase(s), nucleotide analog(s), nucleic acid template(s) and other reaction conditions.

Many such polymerases suitable for the practice of the invention are commercially available, e.g., for use in sequencing, labeling and amplification technologies. For example, human DNA Polymerase Beta is available from R&D systems. DNA polymerase I is available from Epicenter, GE Health Care, Invitrogen, New England Biolabs, Promega, Roche Applied. Science, Sigma Aldrich and many others. The Klenow fragment of DNA Polymerase I is available in both recombinant and protease digested versions, from, e.g., Ambion, Chimera, eEnzyme LLC, GE Health Care, Invitrogen, New England Biolabs, Promega, Roche Applied Science, Sigma Aldrich and many others. ^{Φ}29 DNA polymerase is available from, e.g., Epicentre. Poly A polymerase, reverse transcriptase, Sequenase, SP6 DNA polymerase, T4 DNA polymerase, T7 DNA polymerase, and a variety of thermostable DNA polymerases (Taq, hot start, titanium Taq, etc.) are available from a variety of these and other sources. Recent commercial DNA polymerases include Phusion™. High-Fidelity DNA Polymerase, available from New England Biolabs; GoTaq®Flexi DNA Polymerase, available from Promega; RepliPHI™. ^{Φ}29 DNA Polymerase, available from Epicentre Biotechnologies; PfuUltra™. Hotstart DNA Polymerase, available from Stratagene; KOD HiFi DNA Polymerase, available from Novagen; and many others. Biocompare.com provides comparisons of many different commercially available polymerases.

Available DNA polymerase enzymes have also been modified in any of a variety of ways, e.g., to reduce or eliminate exonuclease activities (many native DNA polymerases have a proof-reading exonuclease function that interferes with, e.g., sequencing applications), to simplify production by making protease digested enzyme fragments such as the Klenow fragment recombinant, etc. Polymerases have also been modified to confer improvements in specificity, processivity, and improved retention time of modified nucleotides in polymerase-DNA-nucleotide complexes (e.g., WO 2007/076057 POLYMERASES FOR NUCLEOTIDE ANALOGUE INCORPORATION by Hanzel et al. and WO 2008/051530 POLYMERASE ENZYMES AND REAGENTS FOR ENHANCED NUCLEIC ACID SEQUENCING by Rank et al.), to alter branch fraction and translocation (e.g., U.S. patent application Ser. No. 12/584,481 filed Sep. 4, 2009, by Pranav Patel et al. entitled “ENGINEERING POLYMERASES AND REACTION CONDITIONS FOR MODIFIED INCORPORATION PROPERTIES”), and to improve surface-immobilized enzyme activities (e.g., WO 2007/075987 ACTIVE SURFACE COUPLED POLYMERASES by Hanzel et al. and WO 2007/076057 PROTEIN ENGINEERING STRATEGIES TO OPTIMIZE ACTIVITY OF SURFACE ATTACHED PROTEINS by Hanzel et al.). Any of these available modified polymerases can be used in the sensor complexes of the present invention.

In certain embodiments, an RNA polymerase may be a suitable polymerase for the practice of the present invention. Suitable RNA polymerases may include any DNA-dependent RNA polymerase or RNA-dependent RNA polymerase. In other embodiments, the polymerase may be a reverse transcriptase (reverse transcription polymerases).

In other embodiments, the polymerases can be further modified for application-specific reasons, such as to reposition amino acid residues used as conjugation sites, e.g., one or more cysteine residues. In one embodiment, a polymerase is modified to reposition a cysteine residue from the palm domain to a finger or thumb domain. In another embodiment, polymerases can be modified to increase mobility, or conformational flexibility, during single nucleotide binding and/or incorporation events so as to enhance signal discrimination.

In order that most of the nucleotides in the target nucleic acid are correctly identified by the molecular sensor complex, the enzyme must process the nucleic acid in a buffer background which is compatible with discrimination of the nucleotides. The enzyme preferably has at least residual activity in a salt concentration well above the normal physiological level, such as from 100 mM to 500 mM. The enzyme is more preferably modified to increase its activity at high salt concentrations. The enzyme may also be modified to improve its processivity, stability and shelf-life.

In yet other embodiments, the polymerase can be altered in a region forming an interface with another component of a molecular sensor complex to optimize assembly of the complex. For example, the amino acids forming an interface can be altered to produce a greater net positive or negative charge at the surface, e.g., to promote ionic interactions with a pore or other component of the sensor complex. Alternatively, amino acids can be altered to reduce the overall net charge at an interface, e.g., to promote hydrophobic interactions with a pore or other component of the sensor complex. In other embodiments, chimeric polymerases made from a mosaic of different sources, e.g., fusion protein, can be used.

Nucleic acids encoding the enzyme can be obtained using routine techniques in the field of recombinant genetics. Basic texts disclosing the general methods of use in this invention include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994-1999). Such nucleic acids may also be obtained through in vitro amplification methods such as those described herein and in Berger, Sambrook, and Ausubel, as well as Mullis et al., (1987) U.S. Pat. No. 4,683,202; PCR Protocols A Guide to Methods and Applications (Innis et al., eds) Academic Press Inc. San Diego, Calif. (1990) (Innis); Arnheim & Levinson (Oct. 1, 1990) C&EN 36-47; The Journal Of NIH Research (1991) 3: 81-94; Kwoh et al. (1989) Proc. Natl. Acad. Sci. USA 86: 1173; Guatelli et al. (1990) Proc. Natl. Acad. Sci. USA 87, 1874; Lomell et al. (1989) J. Clin. Chem., 35: 1826; Landegren et al., (1988) Science 241: 1077-1080; Van Brunt (1990) Biotechnology 8: 291-294; Wu and Wallace (1989) Gene 4: 560; and Barringer et al. (1990) Gene 89: 117, each of which is incorporated by reference in its entirety for all purposes and in particular for all teachings related to amplification methods.

Modifications can additionally be made to the polymerase without diminishing its biological activity. Some modifications may be made to facilitate the cloning, expression, or incorporation of a domain into a protein. Such modifications can include, for example, the addition of codons at either terminus of the polynucleotide that encodes the binding domain to provide, for example, a methionine added at the amino terminus to provide an initiation site, or additional amino acids (e.g., poly His) placed on either terminus to create conveniently located restriction sites or termination codons or purification sequences.

The modified enzymes described herein can be expressed in a variety of host cells, including E. coli, other bacterial hosts, yeasts, filamentous fungi, and various higher eukaryotic cells such as the COS, CHO and HeLa cells lines and myeloma cell lines. Techniques for gene expression in microorganisms are described in, for example, Smith, Gene Expression in Recombinant Microorganisms (Bioprocess Technology, Vol. 22), Marcel Dekker, 1994.

There are many expression systems for producing the modified enzymes described herein that are known to those of ordinary skill in the art. See, e.g., Gene Expression Systems, Fernandex and Hoeffler, Eds. Academic Press, 1999; Sambrook and Russell, supra; and Ausubel et al, supra.) Typically, the polynucleotide that encodes the fusion polypeptide is placed under the control of a promoter that is functional in the desired host cell. Many different promoters are available and known to one of skill in the art, and can be used in the expression vectors of the invention, depending on the particular application. Ordinarily, the promoter selected depends upon the cell in which the promoter is to be active. Other expression control sequences such as ribosome binding sites, transcription termination sites and the like are also optionally included. Constructs that include one or more of these control sequences are termed “expression cassettes.” Accordingly, the nucleic acids that encode the joined polypeptides are incorporated for high level expression in a desired host cell.

Expression control sequences that are suitable for use in a particular host cell are often obtained by cloning a gene that is expressed in that cell. Commonly used prokaryotic control sequences, which are defined herein to include promoters for transcription initiation, optionally with an operator, along with ribosome binding site sequences, include such commonly used promoters as the beta-lactamase (penicillinase) and lactose (lac) promoter systems (Change et al., Nature (1977) 198: 1056), the tryptophan (trp) promoter system (Goeddel et al., Nucleic Acids Res. (1980) .delta.: 4057), the tac promoter (DeBoer, et al., Proc. Natl. Acad. Sci. U.S.A. (1983) 80:21-25); and the lambda-derived PL promoter and N-gene ribosome binding site (Shimatake et al., Nature (1981) 292: 128). The particular promoter system is not critical, any available promoter that functions in prokaryotes can be used. Standard bacterial expression vectors include plasmids such as pBR322-based plasmids, e.g., pBLUESCRIPT™, pSKF, pET23D, lambda-phage derived vectors, and fusion expression systems such as GST and LacZ. Epitope tags can also be added to recombinant proteins to provide convenient methods of isolation, e.g., c-myc, HA-tag, 6-His tag, maltose binding protein, VSV-G tag, anti-DYKDDDDK tag, or any such tag, a large number of which are well known to those of skill in the art.

A variety of protein isolation and detection methods are known and can be used to isolate enzymes, e.g., from recombinant cultures of cells expressing the recombinant enzymes of the invention. A variety of protein isolation and detection methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997); Bioseparation of Proteins, Academic Press, Inc.; Bollag et al. (1996), Satinder Ahuja ed., Handbook of Bioseparations, Academic Press (2000).

While DNA polymerases described herein may have differences in their detailed structure, they generally share the common overall architectural features as described herein, e.g., they have a shape that can be compared with that of a right hand, consisting of “thumb,” “palm,” and “fingers” domains.

### Other Exemplary Processive Nucleic Acid Processing Enzymes

Any enzyme capable of processively processing a nucleic acid molecule while undergoing changes in conformation is suitable for the practice of the present invention. In some embodiments, the enzyme comprises an exonuclease. Exonucleases are enzymes that function by cleaving nucleotides one at a time from the end (exo) of a polynucleotide chain. A hydrolyzing reaction that breaks phosphodiester bonds at either the 3′ or 5′ end occurs. The exonuclease can be a 5′ to 3′ exonuclease or a 3′ to 5′ exonuclease. Suitable exonucleases include, but are not limited, T7 exonuclease, lambda exonuclease, mung bean exonuclease, ExoI, Exo III, Exo IV, ExoVII, exonuclease of Klenow fragment, exonuclease of Poll, Taq exonuclease, T4 exonuclease, etc.

Briefly, exonuclease sequencing determines the sequence of a nucleic acid by degrading the nucleic acid unilaterally from a first end with an exonuclease to sequentially release individual nucleotides. With the processing and sequential release of each nucleotide, a conformational change in the exonuclease occurs and the nucleotide is identified by the corresponding characteristic current modulation as described above. The sequence of the nucleic acid is thus determined from the sequence of conformational changes and current modulation characteristics.

In particular embodiments, a nucleic acid that is sequenced using an exonuclease can contain one or more species of modified nucleotide subunits. Individual species of nucleotide subunits can contain a unique moiety that interacts with an exonuclease during removal from the nucleic acid to produce a type, rate or time duration for a conformational signal change that is distinguishable from the type, rate or time duration produced by the other types of nucleotide species that are removed from the nucleic acid. The nucleic acid can contain at least 1, at least 2, at least 3 or at least 4 modified nucleotide species.

In other embodiments, the processive nucleic acid processing enzyme comprises a helicase. Helicases are enzymes that function by unwinding doubled-stranded DNA or translocating single-stranded DNA using energy derived from ATP hydrolysis. Helicases may assemble to form a ring-shaped structure with six identical protein subunits encircling the target nucleic acid in the channel. Suitable helicases include, but are not limited to, helicases from superfamily 1 or superfamily 2, a Hel308 helicase, a RecD helicase, a Tral helicase, a Tral subgroup helicase, an XPD helicase, etc.

Helicases are dynamic structures that are in constant motion and can therefore exist in several conformation states while controlling the movement of a polynucleotide. Briefly, helicase sequencing determines the sequence of a nucleic acid by unwinding or translocating the nucleic acid unilaterally from a first end with a helicase to sequentially unwind or translocate individual nucleotides. Each of the sequentially unwound or translocated nucleotides is identified by a conformational change in the helicase as it processes the nucleotide and the sequence of the nucleic acid is determined from the sequence of conformational changes and current modulation characteristics as described above.

### Target Nucleic Acids

The target nucleic acids of the invention can comprise any suitable polynucleotide, including double-stranded DNA, single-stranded DNA, single-stranded DNA hairpins, DNA/RNA hybrids, and the like. Further, target nucleic acids may be a specific portion of a genome of a cell, such as a gene, an exon, an intron, a regulatory region, an allele, a variant or mutation; the whole genome; or any portion thereof. The target polynucleotide may be of any length, such as at between about 10 bases and about 100,000 bases, or between about 100 bases and 10,000 bases.

The target nucleic acids of the invention can include unnatural nucleic acids such as PNAs, modified oligonucleotides (e.g., oligonucleotides comprising nucleotides that are not typical to biological RNA or DNA), modified phosphate backbones, modified bases or modifies sugars. A non-natural nucleic acid can be, e.g., single-stranded or double-stranded.

### Reaction Mixtures and Conditions

In general, the reaction mixtures and conditions of the present invention are suitable for nucleic acid sequencing with a molecular sensor complex, i.e., they should enable both activity of the processing enzyme as well as conduct current flow. A reaction mixtures can include one or more nucleotide species.

In certain embodiments relating to sensor complexes comprising a DNA polymerase, a reaction composition or method used for sequence analysis can include four different nucleotide species capable of forming Watson-Crick base pairs with four respective nucleotide species in a nucleic acid template being synthesized. Any of a variety of nucleotide species can be useful in a reaction mixture of a method or composition set forth herein. For example, naturally occurring nucleotides can be used such as dATP, dTTP, dCTP, dGTP, dADP, dTDP, dCDP, dGDP, dAMP, dTMP, dCMP, dGMP, ATP, UTP, CTP, GTP, ADP, UDP, CDP, GDP, AMP, UMP, CMP, and GMP. Typically, dNTP nucleotides are incorporated into a DNA strand by DNA polymerases. In some embodiments, NTP nucleotides or analogs thereof can be incorporated into DNA by a DNA polymerase, for example, in cases where the NTP, or analog thereof, is capable of being incorporated into the DNA by the DNA polymerase and where the conformation or rate or time duration for a DNA polymerase binding and incorporation using the NTP, or analog thereof, can be distinguished from the conformation or rate or time duration for the DNA polymerase binding and incorporation of another nucleotide.

Non-natural nucleotide analogs are also useful. Particularly useful non-natural nucleotide analogs include, but are not limited to, those that produce a detectably different polymerase conformation or rate or time duration for a polymerase incorporation that can be distinguished from the conformation or rate or time duration for a polymerase incorporation of another nucleotide. Other nucleotide analogs that can be used include, but are not limited to, dNTPαS; NTPαS; nucleotides having unnatural nucleobases identified in Hwang et al., Nucl. Acids Res. 34:2037-2045 (2006) (incorporated herein by reference) as ICS, 3MN, 7AI, BEN, DMS, TM, 2Br, 3Br, 4Br, 2CN, 3CN, 4CN, 2FB, 3FB, MM1, MM2 and MM3; or nucleotides having other non-natural nucleobases such as those described in Patro et al. Biochem. 48:180-189 (2009) (incorporated herein by reference) which include 2-amino-1-deazapurine, 1-deazapurine, 2-pyridine, hypoxanthine, purine, 6-Cl-purine, 2-amino-dA, 2-amino purine or 6-Cl-2-amino-purine or nucleotides having non-natural nucleobases such as those described in Krueger et al. Chem Biol. 16:242-8 (2009) (incorporated herein by reference) which include iso-G, iso-C, 5SICS, MMO2, Ds, Pa, FI, FB, dZ, DNB, thymine isosteres, 5-NI, dP, azole-carboxamide, xA, Im-No, Im-ON, J, A*, T*.

In some embodiments, non-natural nucleotide analogs may include analogs in which the heterocyclic base is modified by addition of a chemical moiety that alters the physical properties of the nucleotide without substantially interfering with Watson and Crick base-pairing. In one embodiment, the chemical moiety may be a linear tether molecule comprised of repeats of a monomer, such as PEG. Useful analogs for the practice of the present invention will be those that induce greater conformational changes in the enzyme upon single nucleic acid processing events. In yet other embodiments, non-natural nucleotide analogs may include analogs in which the alpha phosphate is modified by addition of a chemical moiety that alters the physical properties of the nucleotide without interfering with Watson and Crick base-pairing. Examples of suitable chemical moieties are those disclosed, e.g., in Vaghefi M 2005, Nucleoside Triphosphates and their Analogs: Chemistry, Biotechnology, and Biological Applications, CRC Press, Boca Raton, Fla. (incorporated herein by reference).

The enzyme reaction conditions include, e.g., the type and concentration of buffer, the pH of the reaction, the temperature, the type and concentration of salts, the presence of particular additives which influence the kinetics of the enzyme, and the type, concentration, and relative amounts of various cofactors, including metal cofactors.

Enzymatic reactions are often run in the presence of a buffer, which is used to control the pH of the reaction mixture. The type of buffer can in some cases influence the kinetics of the polymerase reaction. For example, in some cases, use of TRIS as buffer is useful. Suitable buffers include, for example, TAPS (3-{[tris(hydroxymethyl)methyl]amino}propanesulfonic acid), Bicine (N,N-bis(2-hydroxyethyl)glycine), TRIS (tris(hydroxymethyl)methylamine), ACES (N-(2-Acetamido)-2-aminoethanesulfonic acid), Tricine (N-tris(hydroxymethyl)methylglycine), HEPES 4-2-hydroxyethyl-1-piperazineethanesulfonic acid), TES (2-{[tris(hydroxymethyl)methyl]amino}ethanesulfonic acid), MOPS (3-(N-morpholino)propanesulfonic acid), PIPES (piperazine-N,N′-bis(2-ethanesulfonic acid)), and MES (2-(N-morpholino)ethanesulfonic acid).

The pH of the reaction can influence the kinetics of the polymerase reaction. The pH can be adjusted to a value that optimizes enzyme activity in transmembrane pore compatible buffers. The pH is generally between about 6 and about 9. In some cases, the pH is between about 6.5 and about 8.0. In some cases, the pH is between about 6.5 and 7.5. In some cases, the pH is about 7.4. For the practice of the present invention, it is important that the pH of the buffer suitably maintains the stability and function of both the membrane and pore.

The temperature of the reaction can be adjusted. The reaction temperature may depend upon the type of polymerase which is employed. Temperatures should be compatible with the type of membrane and transmembrane pore employed. Temperatures between 15° C. and 90° C., between 20° C. and 50° C., between 20° C. and 40° C., or between 20° C. and 30° C. can be used. In some embodiments, the temperature is preferably about 20° C.

The ionic strength of the solution can be tailored to optimize current flow and minimize the measured background in order to improve the ability to measure the current blockage. The reaction conditions can also be modified to optimize enzyme activity in high salt buffers. In particular, the ionic strength can be adjusted using small ions in order to obtain suitable enzyme activity and pore current. In certain embodiments, small ions may be provided by salts such as NH₄OAc and NH₄Cl.

In some cases, additives and/or cofactors can be added to the reaction mixture to optimize enzyme activity. Suitable cofactors for the enzymes of the present invention may include MgCl₂ and MnCl₂. In some cases, the additives can interact with the active site of the enzyme, acting for example as competitive inhibitors. In some cases, additives can interact with portions of the enzyme away from the active site in a manner that will optimize activity and/or stability in transmembrane pore compatible buffers. Additives suitable for the practice of the present invention may include PEG, DMSO, and the like.

### Localization and Attachment Structures

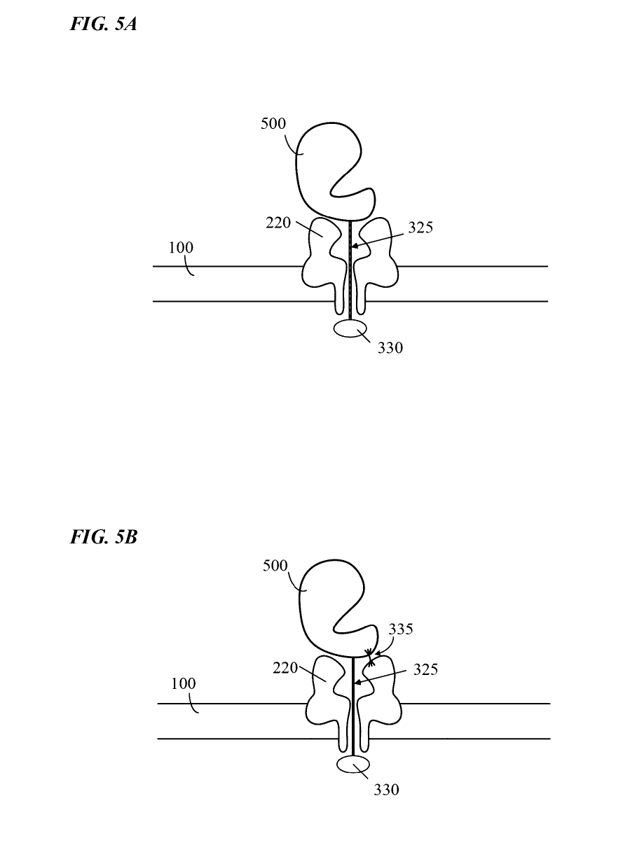

For proper function of the molecular sensor complexes of the present invention, it is necessary that the enzyme be stably localized to the pore in sufficiently close proximity to reliably influence, or modulate, current flow through the pore. Several alternative localization and/or attachment structures or compositions are contemplated by the present invention, some which are illustrated schematically in FIGS. 5-8. FIG. 5A depicts one embodiment in which enzyme 500 is localized to pore 220 by covalent attachment to tethering structure 325, herein referred to simply as a “tether”. Tethers may be designed to thread through the lumen of the pore, from one side of membrane 100 to the other. Tethers may comprise one or more structural domains, or “segments”, designed to perform one or more functions. In one embodiment, a tether is constructed of three domains: 1) a polyethylene glycol (PEG) repeat segment, located proximal to the enzyme and designed to span the pore lumen; 2) a short oligonucleotide, designed to hybridize to a single-stranded oligonucleotide on the opposite side of the nanopore relative to the enzyme; and 3) a negatively charged phosphoramidite tail, located most distal to the enzyme and designed to facilitate threading of the tether through the pore. Other alternatives to PEG (i.e., repeat moieties suitable for the pore spanning segment) and to phosphoramidites (i.e., negatively charged moieties suitable for the tail segment) will be appreciated by the skilled artisan. The tether may be retained in the pore by anchoring structure 330, herein referred to simply as “anchor”, located on the distal side of the pore relative to the enzyme. The anchor may be formed by any molecular structure with a diameter larger than that of the pore. In one embodiment, the anchor is a double-stranded oligonucleotide formed by hybridizing a complementary single-stranded oligonucleotide to the oligonucleotide domain in the tether. In other embodiments, the anchor may be formed a complex of biotin and streptavidin.

Tethers may include one or more modifications for application-specific reasons. In one embodiment, tethers are modified to optimize nucleotide-specific current modulation characteristics. Certain embodiments involve placing at least one nucleotide, such as dTTP, within the region of the tether that spans the channel of the pore, e.g., within the PEG repeat region described above.

A method of attaching the tether to the enzyme is via cysteine linkage. This can be mediated by a bi-functional chemical linker or by a polypeptide linker with a terminal presented cysteine residue. Cysteines can be introduced at various positions, as disclosed herein. The length, reactivity, specificity, rigidity and solubility of any bi-functional linker may be designed to ensure that the enzyme is positioned correctly in relation to the pore and the function of both the enzyme and pore is retained. Suitable linkers include disulfide linkers such as 2,2′ dithiodipyridine and bismaleimide crosslinkers, such as 1,4-bis(maleimido)butane (BMB) or bis(maleimido)hexane. One drawback of bi-functional linkers is the requirement of the enzyme to contain no further surface accessible cysteine residues, as binding of the bi-functional linker to these cannot be controlled and may affect substrate binding or activity. If the enzyme does contain several accessible cysteine residues, modification of the enzyme may be required to remove them while ensuring the modifications do not affect the folding or activity of the enzyme. The reactivity of cysteine residues may be enhanced by modification of the adjacent residues, for example on a peptide linker. For instance, the basic groups of flanking arginine, histidine or lysine residues will change the pKa of the cysteines thiol group to that of the more reactive S⁻ group. The reactivity of cysteine residues may be protected by thiol protective groups such as dTNB. These may be reacted with one or more cysteine residues of the enzyme or subunit, either as a monomer or part of an oligomer, before a linker is attached.

FIG. 5B depicts an alternative embodiment of the invention in which enzyme 500 is directly attached to pore 220, e.g., by one or more covalent attachments 335. In such configurations, the current conducting abilities of the pore are retained or optimized. Similarly, the activity of the enzyme, which is typically provided by its secondary structural elements (α-helices and β-strands) and tertiary structural elements, is not compromised. In order to avoid diminishing sensor complex function, the sites of attachment are preferably residues or regions in the pore and enzyme that do not affect secondary or tertiary structure. Suitable configurations include, but are not limited to, the amino terminus of the pore being attached to the carboxy terminus of the enzyme and vice versa. Alternatively, the two components may be attached via amino acids within their sequences. For instance, the enzyme may be attached to one or more amino acids on the surface of the pore proximal to its top opening. The enzyme may be attached to the pore at more than one, such as two or three, points. Attaching the enzyme to the pore at more than one point can be used to constrain the mobility of the enzyme. For instance, multiple attachments may be used to constrain the freedom of the enzyme to rotate or its ability to move away from the pore. The enzyme can be attached to the pore with any suitable chemistry (e.g., covalent bond and/or linker).

In some cases, the enzyme is attached to the pore with molecular staples. In some instances, molecular staples comprise three amino acid sequences (denoted linkers A, B and C). Linker A can extend from the pore, Linker B can extend from the enzyme, and Linker C then can bind Linkers A and B (e.g., by wrapping around both Linkers A and B) and thus the enzyme to the pore. Linker C can also be constructed to be part of Linker A or Linker B, thus reducing the number of linker molecules. Linkers may also be biotin and streptavidin.

The enzyme may be attached to the pore using one or more, such as two or three, linkers. The one or more linkers may be designed to constrain the mobility of the enzyme. The linkers may be attached to one or more reactive cysteine residues, reactive lysine residues or non-natural amino acids in the pore and/or enzyme. Suitable linkers are well-known in the art. Suitable linkers include, but are not limited to, chemical crosslinkers and peptide linkers. Preferred linkers are amino acid sequences (i.e., peptide linkers). The length, flexibility and hydrophilicity of the peptide linker are typically designed such that it does not to disturb the functions of the enzyme and pore. Preferred flexible peptide linkers are stretches of 2 to 20, such as 4, 6, 8, 10 or 16, serine and/or glycine amino acids. More preferred flexible linkers include (SG)₁, (SG)₂, (SG)₃, (SG)₄, (SG)₅ and (SG)₈ wherein S is serine and G is glycine. Preferred rigid linkers are stretches of 2 to 30, such as 4, 6, 8, 16 or 24, proline amino acids. More preferred rigid linkers include (P)₁₂ wherein P is proline.

In some instances, the enzyme is linked to the pore using Solulink™ chemistry. Solulink™ can be a reaction between HyNic (6-quadrature-hydrazino-quadrature-nicotinic acid, an aromatic hydrazine) and 4FB (4-formylbenzoate, an aromatic aldehyde). In some instances, the enzyme is linked to the pore using Click chemistry (available from LifeTechnologies for example). In some cases, mutations are introduced into the pore molecule and then a molecule is used (e.g., a DNA intermediate molecule) to link the enzyme to the mutation sites on the pore.

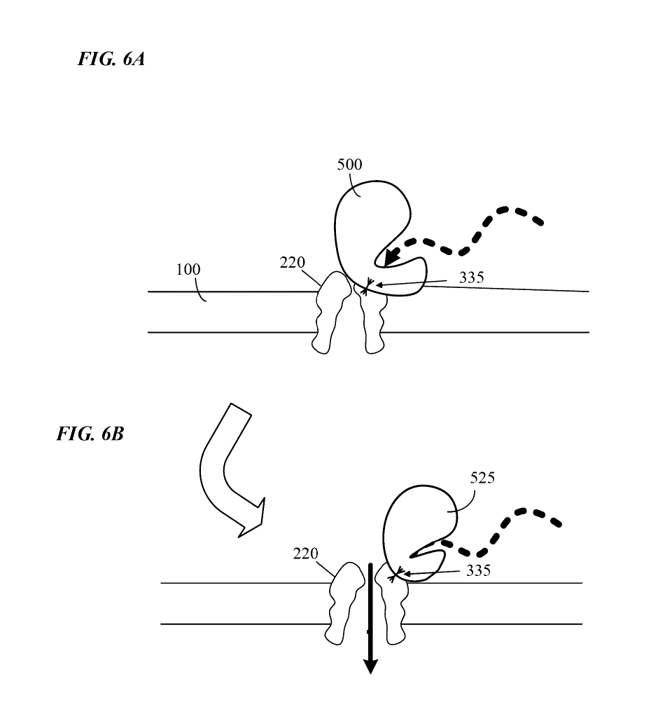

As depicted in FIG. 6A, enzyme 500 may in certain embodiments be directly attached to transmembrane pore 220 by covalent linker 335 in the absence of a pore-threading tether. This mechanism of attachment is useful in sensor complex constructs in which the lowest free energy state of the pore favors a “closed” configuration. As further illustrated in FIG. 6B, conformational change 525 in the enzyme, triggered by a nucleic acid processing event, shifts the pore to an open configuration, which increases the current flow through the pore.

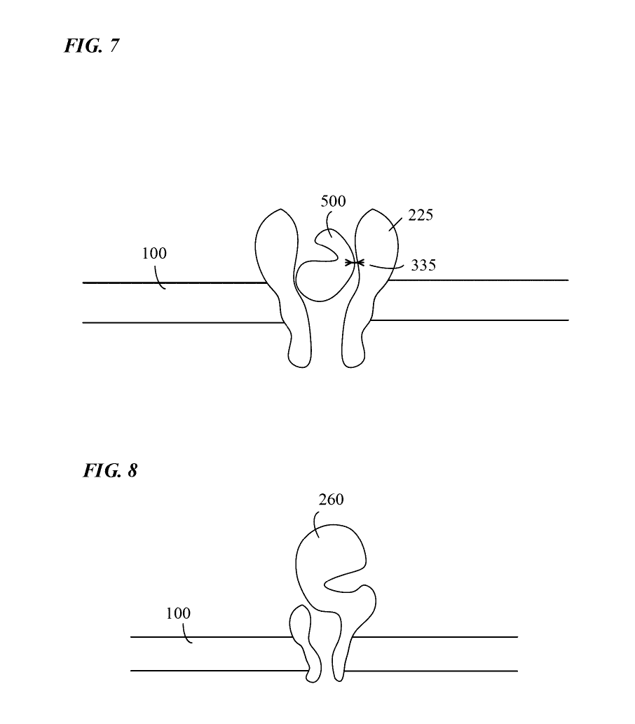

In another embodiment illustrated in FIG. 7, enzyme 500 is localized within the channel of pore 225 by covalent attachment via one or more linker 335. In this configuration, conformational changes in the enzyme modulate current flow through the pore in a nucleotide-dependent manner, as described elsewhere herein. The size of the enzyme and/or the pore channel may be altered to optimize the physical dimensions of the protein(s) to accommodate this configuration. For example, regions or domains of the protein(s) not essential for sensor complex function may be removed. In an alternative embodiment, the enzyme and the pore are expressed as a chimeric, or fusion protein to localize the enzyme within the channel of the pore, as described below.

In the embodiment illustrated in FIG. 8, the enzyme and pore are expressed as chimeric complex 260, which may be a fusion protein. In some embodiments, the coding sequences of each polypeptide in a resulting fusion protein (e.g., the enzyme and the pore) are directly joined at their amino- or carboxy-terminus via a peptide bond. Alternatively, an amino acid linker sequence may be employed to separate the first and second polypeptide components by a distance sufficient to ensure that each polypeptide folds into its secondary and tertiary structures. Such an amino acid linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Typical peptide linker sequences contain Gly, Ser, Val and Thr residues. Other near neutral amino acids, such as Ala can also be used in the linker sequence. Amino acid sequences which may be usefully employed as linkers include those disclosed in Maratea et al. (1985) Gene 40:39-46; Murphy et al. (1986) Proc. Natl. Acad. Sci. USA 83:8258-8262; U.S. Pat. Nos. 4,935,233 and 4,751,180, each of which is hereby incorporated by reference in its entirety for all purposes and in particular for all teachings related to linkers. The linker sequence may generally be from 1 to about 50 amino acids in length, e.g., 3, 4, 6, or 10 amino acids in length, but can be 100 or 200 amino acids in length. Linker sequences may not be required when the first and second polypeptides have non-essential N-terminal amino acid regions that can be used to separate the functional domains and prevent steric interference.

Other chemical linkers include carbohydrate linkers, lipid linkers, fatty acid linkers, polyether linkers, e.g., PEG, etc. For example, poly(ethylene glycol) linkers are available from Shearwater Polymers, Inc. Huntsville, Ala. These linkers optionally have amide linkages, sulfhydryl linkages, or heterobifunctional linkages.

In another embodiment, the molecular sensor complex may include a single enzyme linked to more than a single nanopore. In one embodiment, a single enzyme is separately linked to each of two different nanopores. The nanopores may be in a normally “closed” configuration that transitions to an “open” (i.e., current conducting) configuration upon enzyme movement induced by single nucleic acid processing events. In this manner, electronic signals induced by conformational changes in the enzyme may be effectively amplified.

### Methods of Assembling Molecular Sensor Complexes

The present invention also provides methods of assembling, or producing, molecular sensor complexes of the invention. The molecular sensor complexes may be formed by allowing at least one component of the invention to assemble with other suitable subunits or by covalently attaching an enzyme to a tether or region of a transmembrane pore, as discussed above. Any of the constructs, subunits, enzymes or pores discussed above can be used in the methods. The site of and method of covalent attachment are selected as discussed above. In one embodiment, a sensor complex is assembled with an enzyme-tether conjugate. The negative charge of the phosphoramidite tail of the tether is used to draw the conjugate to the pore upon application of an external voltage. The tether is able to thread through the pore and localize the enzyme to the pore opening. The enzyme-tether conjugate may be secured in place by addition of an oligonucleotide anchor to the trans side of the membrane, as described herein.

In some embodiments, the target, or substrate, nucleic acid may be preloaded onto the processive nucleic acid processing enzyme before the enzyme is localized to the nanopore. In other embodiments, the nucleic acid may be loaded onto the enzyme after the enzyme is localized to the nanopore. The skilled artisan will appreciate that the manner of loading the nucleic acid template will be influenced by the particular template and enzyme comprising the molecular senor complex.

The methods also comprise determining whether or not the molecular sensor complex is capable of processing nucleic acids and detecting nucleotides. The molecular sensor complex may be assessed for its ability to detect individual nucleotides. Assays for doing this are described herein. If the molecular sensor complex is capable of processing nucleic acids and detecting nucleotides, the pore and enzyme have been attached correctly and a pore of the invention has been produced. If a molecular sensor complex cannot handle nucleic acids and detect nucleotides, a pore and enzyme of the invention have not been produced.

### Methods of Purifying Molecular Sensor Complexes

The present invention also provides methods of purifying molecular sensor complexes of the invention. The methods allow the purification of molecular sensor complexes comprising at least one construct of the invention. The methods do not involve the use of anionic surfactants, such as sodium dodecyl sulphate (SDS), and therefore avoid any detrimental effects on the enzyme part of the construct. The methods are particularly good for purifying molecular sensor complexes comprising a construct of the invention in which the subunit and enzyme have been genetically fused.

The methods involve providing at least one construct of the invention and any remaining subunits required to form a molecular sensor complex of the invention. Any of the constructs and subunits discussed above can be used. Any of the protein subunits may be purified by well-known technologies based on, e.g., his-tagged labeled proteins and Ni-NTA columns. In particular embodiments, the construct(s) and remaining subunits may be inserted into synthetic lipid vesicles and allowed to oligomerize. Methods for inserting the construct(s) and remaining subunits into synthetic vesicles are well known in the art. The vesicles may comprise any components and are typically made of a blend of lipids. Suitable lipids are well-known in the art. The synthetic vesicles may comprise 30% cholesterol, 30% phosphatidylcholine (PC), 20% phosphatidylethanolamine (PE), 10% sphingomyelin (SM) and 10% phosphatidylserine (PS). The vesicles may then be contacted with a non-ionic surfactant or a blend of non-ionic surfactants. The non-ionic surfactant may be an Octyl Glucoside (OG) or DoDecyl Maltoside (DDM) detergent. The oligomerized pores may then purified, for example by using affinity purification based on his-tag/Ni-NTA interactions.

### Apparatus and Systems

The methods of the invention may be carried out using any apparatus that is suitable for investigating a molecular sensor complex comprising a pore of the invention inserted into a membrane. The methods may be carried out using any apparatus that is suitable for stochastic sensing. For example, an apparatus comprising a chamber comprising an aqueous solution and a barrier that separates the chamber into two sections. The barrier may have an aperture in which the membrane containing the complex is formed. The nucleotide or nucleic acid may be contacted with the complex by introducing the nucleic acid into the chamber. The nucleic acid may be introduced into either of the two sections of the chamber, but must be introduced into the section of the chamber containing the enzyme. Other components of the sensor complex, such as anchoring members, may be introduced into the section of the chamber opposite the enzyme.

The methods involve measuring the current passing through the pore during enzymatic processing of the target nucleic acid. Therefore the apparatus also comprises an electrical circuit capable of applying a potential and measuring an electrical signal across the membrane and pore. The methods may be carried out using a patch clamp or a voltage clamp. The method preferably involves the use of a voltage clamp.

The methods of the invention involve the measuring of a current passing through the pore during enzymatic processing of the target nucleic acid. Suitable conditions for measuring ionic currents through transmembrane protein pores are known in the art and disclosed herein. The method is carried out with a voltage applied across the membrane and pore, also referred to herein as a “voltage drop”. The voltage used is typically from −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more preferably in the range 120 mV to 170 mV. It is possible to increase discrimination between different nucleotides processed by a complex of the invention by using an increased applied potential. In some cases, an AC voltage or a time variable voltage waveform may be applied either in combination with a DC voltage or not.

The methods are carried out in the presence of any alkali metal chloride, acetate, or mixture of chloride and acetate salt. In the exemplary apparatus discussed above, the salt is present in the aqueous solution in the chamber. Potassium chloride (KCl), sodium chloride (NaCl) or ammonium chloride (NH₄Cl) is typically used. KCl or NH₄Cl is preferred. The salt concentration is typically from 0.1 to 2.5M, from 0.3 to 1.9M, from 0.5 to 1.8M, from 0.7 to 1.7M, from 0.9 to 1.6M or from 1M to 1.4M. High salt concentrations provide a high signal to noise ratio and allow for currents indicative of the presence of a nucleotide to be identified against the background of normal current fluctuations. However, lower salt concentrations are preferably used so that the enzyme is capable of functioning. The salt concentration is preferably from 150 to 500 mM. Good signal distinction at these low salt concentrations can be achieved by carrying out the method at temperatures above room temperature, such as from 30° C. to 40° C.

In addition to increasing the solution temperature, there are a number of other strategies that can be employed to increase the conductance of the solution, while maintaining conditions that are suitable for enzyme activity. One such strategy is to use the lipid bilayer to divide two different concentrations of salt solution, a low salt concentration of salt on the enzyme side and a higher concentration on the opposite side as described, e.g., in the Examples.

The invention relates in some aspects to systems for sequencing with molecular sensor complexes. In some cases, the systems comprise devices with resistive openings between fluid regions in contact with the sensor complex and fluid regions which house a drive electrode. The devices of the invention can be made using a semiconductor substrate such as silicon to allow for incorporated electronic circuitry to be located near each pore of a complex. The devices of the invention will therefore comprise arrays of both microfluidic and electronic elements. In some cases, the semiconductor which has the electronic elements also includes microfluidic elements that contain the sensor complexes. In some cases, the semiconductor having the electronic elements is bonded to another layer which has incorporated microfluidic elements that contain the sensor complexes.

The devices of the invention generally comprise a microfluidic element into which a sensor complex is disposed. This microfluidic element will generally provide for fluid regions on either side of the sensor complex through which the ion current to be detected for sequence determination will pass as described above. In some cases, the fluid regions on either side of the sensor complex are referred to as the cis and trans regions, where ion current generally travels from the cis region to the trans region through the pore. For the purposes of description, the terms upper and lower are also used to describe such reservoirs and other fluid regions. It is to be understood that the terms upper and lower are used as relative rather than absolute terms, and in some cases, the upper and lower regions may be in the same plane of the device. The upper and lower fluidic regions are electrically connected either by direct contact, or by fluidic (ionic) contact with drive and measurement electrodes. In some cases, the upper and lower fluid regions extend through a substrate, in other cases, the upper and lower fluid regions are disposed within a layer, for example, where both the upper and lower fluidic regions open to the same surface of a substrate. Methods for semiconductor and microfluidic fabrication described herein and as known in the art can be employed to fabricate the devices of the invention.

The invention involves the use of a current sensing circuit used to measure the ion current that is modulated by the enzyme conformational changes. The circuit measures the ion current passing through the reaction mixture (typically comprising, e.g., >1M KCl electrolyte) between two ion sensitive electrodes. The electrodes (e.g., Ag/AgCl electrodes) complete the circuit through a transimpedance amplifier, which provides a voltage output proportional to the ion current across a frequency range. In some embodiments, an array of transimpedance amps implemented in CMOS are arranged to measure an array of independent sensor currents in parallel. An example of such an amplifier array has been disclosed by Kim et al. (see, e.g., (Kim, B. N., Herbst, A. D., Kim, S. J., Minch, B. A., & Lindau, M. 2013. Parallel Recording of Neurotransmitters Release from Chromaffin Cells using a 10×10 CMOS IC Potentiostat Array with On-Chip Working Electrodes. Biosensors and Bioelectronics, 41, 736-744).

Systems of the invention may include a computer, which may implement, control, and/or regulate the voltage of a voltage source, measurements of an ammeter, and display of the ionic current graphs as discussed herein.

Various methods, procedures, circuits, elements, and techniques discussed herein may also incorporate and/or utilize the capabilities of a computer. Moreover, capabilities of a computer may be utilized to implement features of exemplary embodiments discussed herein. One or more of the capabilities of the computer may be utilized to implement, to connect to, and/or to support any element discussed herein (as understood by one skilled in the art) and in FIGS. 1 and 2. For example, the computer may be any type of computing device and/or test equipment (including ammeters, voltage sources, connectors, etc.). An input/output device (having proper software and hardware) of a computer may include and/or be coupled to the molecular sensor complex apparatus discussed herein via cables, plugs, wires, electrodes, patch clamps, etc. Also, the communication interface of the input/output devices comprises hardware and software for communicating with, operatively connecting to, reading, and/or controlling voltage sources, ammeters, and current traces (e.g., magnitude and time duration of current), etc., as discussed herein. The user interfaces of the input/output device may include, e.g., a track ball, mouse, pointing device, keyboard, touch screen, etc., for interacting with the computer, such as inputting information, making selections, independently controlling different voltages sources, and/or displaying, viewing and recording current traces for each base, molecule, biomolecules, etc.

### EXAMPLES

### Example 1

### Assembly of a Polymerase-Based Molecular Sensor Complex

This example demonstrates assembly of a sensor complex incorporating the Klenow Fragment of DNA polymerase I (KF) and the αHL nanopore. In this example, the polymerase was localized to the nanopore by covalent attachment to a tether construct designed to thread through the nanopore and lock into place by hybridization with a short oligonucleotide anchor on the distal side of the nanopore. KF-tether conjugates were generated by labeling the single native cysteine in the palm region of the polymerase; the cysteine was first activated with 2, 2′-dipyridyldisulfide to form a disulfide conjugate and then conjugated with a reduced sulfhydryl-labeled tether construct. The structure of the tethers used in this Example are set forth in Table 1.

TABLE 1

| | PEG | Oligonucleotide | phosphoramidite |
|---|---|---|---|
| tether | repeats | (5′-3′) | (L) tail repeats |
| 1 | 7 | TCAGGTGC | 34 |
| 2 | 4 | TCAGGTGC | 34 |
| 3 | 11 | TCAGGTGC | 34 |

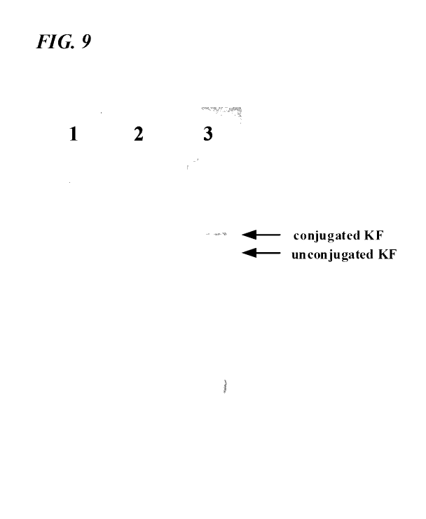

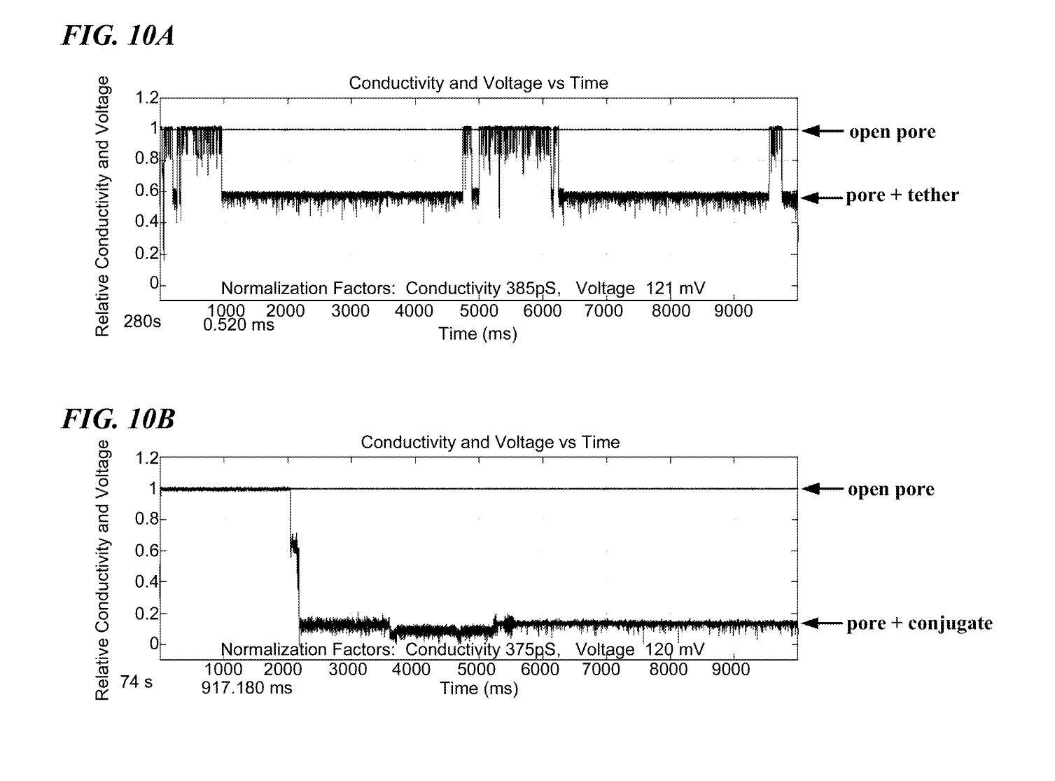

The tethers were constructed of three domains (i.e., “segments”): 1) a polyethylene glycol (PEG) repeat region, located proximal to the polymerase and designed to span the nanopore channel; 2) a short oligonucleotide, designed to hybridize to a single-stranded oligonucleotide on the opposite side of the nanopore relative to the polymerase to anchor the assembly; and 3) a negatively charged phosphoramidite tail, located most distal to the polymerase and designed to facilitate threading of the tether through the nanopore. FIG. 9 is a SDS/PAGE gel that shows the size of the unmodified KF polymerase (lane 1), the KF-tether 1 conjugate (lane 2) and the KF-tether 2 conjugate (lane 3). As expected, the conjugates show an increase in mass compared to the unmodified polymerase.

As a first step to characterize the signature electrical trace of the sensor complex, the effects of tether alone on the flow of current through the nanopore was investigated. To summarize the experimental setup, a lipid bilayer membrane is formed with the lipid 1,2-diphytanoyl-sn-glycero-3-phosphoethanolamine (C₄₅H₉₀NO₈P) across an aperture in a PTFE solid support cell by i) priming the support cell with a thin coat of lipid dissolved in hexane, ii) air-drying the painted cell to remove the hexane iii) painting lipid over the support cell by dissolving PE in 1-hexadecene and depositing the solution over the primed support cell with a pipette and iv) moving an air bubble over the aperture in the support cell to form a lipid bilayer membrane over the aperture. Next, an aqueous solution of the nanopore protein, e.g., αHL, is added to the lipid bilayer and the pore is allowed to self-assemble on the membrane and insert to form a transmembrane electroconductive pore. The lipid bilayer membrane separates the cis and trans reservoirs in the PTFE support cell, each of which contain a Ag/AgCl electrode that are connected to the headstage of the Axopatch 2000B transimpedance amplifier. In this circuit, the Molecular Devices Axopatch 200B instrument applies the voltage between the electrodes, amplifies the resulting ion current passing through the nanopore/membrane and filters the signal at 10 kHz filter. The signal is then digitally sampled at 100 k samples/s and stored for analysis. For this experiment, a sample of tether and short oligonucleotide anchor (e.g., GCACCTGA) was added to a αHL nanopore immobilized in a membrane. The short oligonucleotide was designed to hybridize to the oligonucleotide in the tether, creating a double stranded anchor to immobilize the tether in the nanopore. Conditions for conductivity were set by immersing the membrane in 300 mM NH4OAc on the cis side and 1000 mM NH4Cl on the trans side with the temperature maintained at 20° C. A current of 120 mV was applied and conductivity through the membrane was measured over a 10 second time interval.

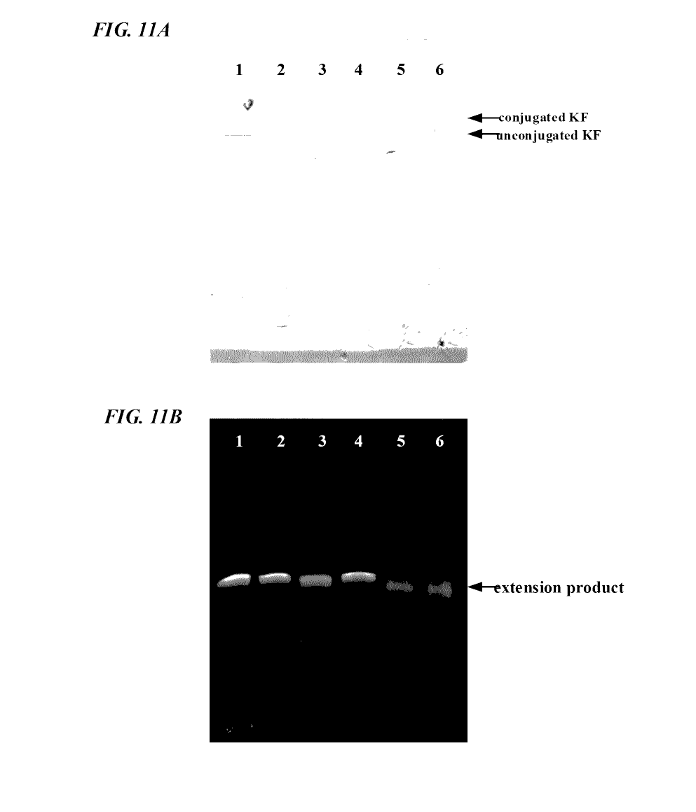

FIG. 10A shows a representative electrical trace, illustrating the dynamics of the nanopore/tether/oligonucleotide assembly over time. Two conductivity levels were observed: baseline and an approximately 40% reduction from baseline, reflecting current flow through the unobstructed nanopore and current flow through the pore threaded with oligonucleotide-immobilized tether, respectively. These signals are transiently stable and reproducible, indicating that the tether alone contributes a measurable electric signal. With time, the current cycles between to these two levels, which likely reflects formation and disruption of the complex as oligonucleotide anchors disassociate and new tether-oligonucleotide complexes thread through the nanopore.

Next, the signature electrical trace of a polymerase-tether conjugate threaded through the nanopore was investigated, under the same conditions described above. The polymerase sensor complex was first assembled by driving the negatively charged polymerase-tether conjugate to the membrane-embedded αHL nanopore by application of an external voltage bias to the cis side of the membrane. Again, the conjugate was secured to the pore with a short oligonucleotide anchor on the trans side of the membrane. FIG. 10B shows a representative electrical trace, indicating two distinct signals: the baseline current flow and a nearly complete reduction in conductance as the polymerase-tether conjugate anchors to the nanopore and physically occludes the opening. That this reduction in signal reflects the assembly of a polymerase-nanopore complex is corroborated by the observation that reversal of the signal back to baseline requires the same voltage level that would be predicted to disassociate the short oligonucleotide anchor from the tether (data not shown). These results indicate that a tether and a tether-polymerase conjugate can be anchored to a nanopore and, moreover, that the resulting complex can generate reproducible electrical signals. Polymerase-nanopore complexes are thus capable of modulating current flow through the pore and show promise as useful sensors to transduct mechanical events into electrical signals.

### Example 2

### Klenow Fragment Variant with Repositioned Conjugation Site

This example describes the generation and preliminary characterization of a KF polymerase variant in which the cysteine conjugation residue was repositioned from the stationary palm domain to the flexible finger domain by a C907S in combination with either a L790C or a S428C amino acid substitutions. The rationale behind this variant was that attachment via a mobile domain might increase the sensitivity of the sensor complex to mechanical movement as the polymerase binds substrates. As a first step in characterizing the variant, the impact of the mutations on polymerase activity were investigated. The KF mutant was conjugated to one of four tether constructs, as described above. In addition to the tethers set forth in Table 1, a fourth tether in which a single nucleotide (T) was engineered into the PEG repeat motif was used. Conjugation was assessed by SDS/PAGE analysis of the polymerase conjugates. As shown in FIG. 11A, the KF mutant (lane 2) was successfully conjugated to each tether construct (lanes 3-6). Next, the extension activity of each conjugate was assessed by a standard in vitro DNA polymerization assay using a labeled primer and singled stranded template. FIG. 11B is a representative gel analysis of the reaction products of each KF mutant conjugate. As can be seen, the unconjugated KF mutant (lane 2) as well as each mutant conjugate (lanes 3-6) exhibited extension activity similar to that of the wildtype polymerase (lanes 1), indicating repositioning of the conjugation site does not compromise function in these reactions.

### Example 3

### Optimization of Polymerase Activity in High-Salt Buffers

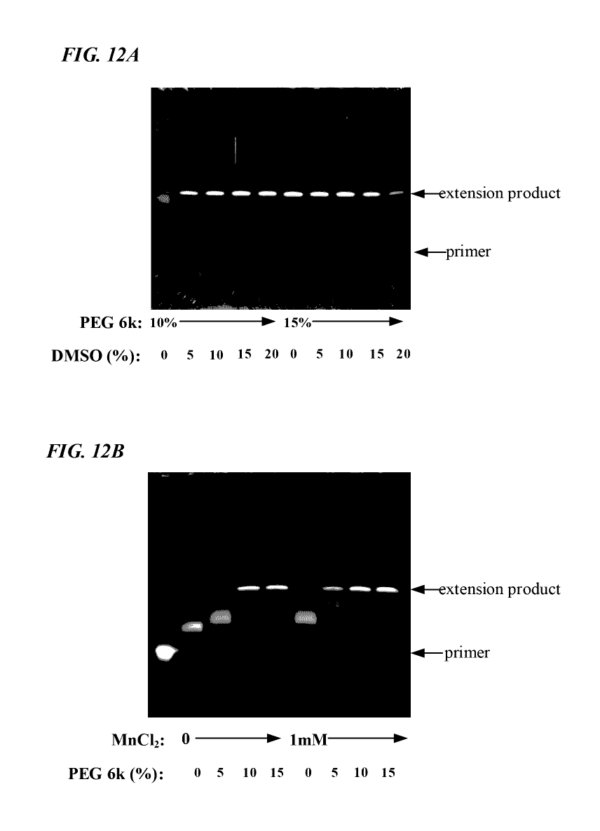

To optimize extension activity, the activity of the Klenow fragment (KF) in a variety of nanopore-compatible reaction conditions was investigated. The base reaction conditions were 750 mM NH₄Cl, 10 mM HEPES, pH 7.4, 10 mM MgCl₂, 1 mM TCEP, 10 mM MnCl₂. Variables tested included the amount of PEG 6k (10% or 15%) and DMSO (0%, 5%, 10%, or 20%) additives. Extension of a labeled 21mer primer hybridized to a short template was carried out for 10 minutes at 20° C. for each reaction and products were analyzed by standard gel electrophoresis. As shown in FIG. 12A, the KF tolerates a broad range of additive levels, though optimal extension activity appears to occur with higher levels of PEG combined with lower levels of DMSO.

Next, the effect of different levels of MnCl₂ and PEG 6k on extension activity in a high salt buffer was investigated. In this experiment, the base reaction conditions were 1M NH₄OAc, 10 mM HEPES, pH 7.4, 10 mM MgCl₂, and 1 mM TCEP. Variables tested were MnCl₂ (none or 1 mM) and PEG 6k (0, 5%, 10%, or 15%). As above, extension of a labeled 21mer primer hybridized to a short template was carried out for 10 minutes at 20° C. for each reaction and products were analyzed by standard gel electrophoresis. As shown in FIG. 12B, optimal extension activity is observed in the presence of 1 mM MnCl₂ and higher levels of PEG 6k. Together, these data indicate that the KF exhibits significant in vitro polymerization activity that can be optimized under nanopore-compatible conditions, including high salt and relatively low pH and temperature, with additives such as PEG 6k and DMSO.

### Example 4

### Assembly and Use of a Molecular Sensor Complex Based on a DNA Exonuclease Nucleic Acid Processing Enzyme

This Example describes how a DNA exonuclease may be assimilated with a nanopore embedded in a lipid bilayer membrane to form a sensor complex for DNA sequencing applications. In this Example, the exonuclease is the phage lambda DNA exonuclease with inherent 5′ to 3′ exonuclease activities. First, a lipid bilayer membrane is formed with the lipid 1,2-diphytanoyl-sn-glycero-3-phosphoethanolamine (C45H90NO8P). Briefly, the lipid bilayer is formed across an aperture in a PTFE solid support cell by priming the cell with a thin coat of lipid dissolved in hexane and coating over the support cell. Hexane is removed by air-drying the painted cell and a lipid membrane is painted over the support cell by dissolving PE in 1-hexadecene and depositing the solution over the primed support cell with a pipette and moving an air bubble over the aperture in the support cell to form a lipid bilayer membrane over the aperture. Next, an aqueous solution of the nanopore protein, e.g., αHL, is added to the lipid bilayer and the pore is allowed to self-assemble on the membrane and insert to form a transmembrane ion conductive pore.

An exonuclease-DNA template complex is next generated. In this Example, the phage lambda DNA exonuclease and double-stranded DNA template are produced using standard molecular biology technologies. In this example, the exonuclease is modified by covalent attachment of a tether construct, as described in Example 1. The 5′ ends of the double-stranded DNA template are phosphorylated using well-known T4 Polynucleotide Kinase based methods. The resulting modified template is purified to using well-known silica glass fiber methods. The double-stranded DNA template is incubated with the phage lambda DNA exonuclease in an aqueous solution containing 30 mM Tris-HCl, pH 7.5, 2 mM EDTA, 4 mM DTT, and 30 mM ammonium acetate, which binds the DNA template following its natural functions but does not initiate exonuclease digestion due to the lack of magnesium cofactor. The lambda exonuclease-DNA template assembly is then assimilated, or coupled, with the nanopore embedded in the lipid bilayer membrane by adding the assembly to the cis reservoir of the nanopore sensor containing an aqueous solution of 30 mM Tris-HCl, pH 7.5, 2 mM EDTA, 4 mM Dithiothreitol, and 300 mM Ammonium Acetate in the cis reservoir and an aqueous solution of 1000 mM NH4Cl on the trans side reservoir. An electric potential is applied across the membrane to thread the negatively charged tether through the pore, thereby guiding and the DNA exonuclease complex to the nanopore. The exonuclease is secured to the nanopore by hybridizing a short oligonucleotide anchor to the tether construct on the distal side of the nanopore.

While maintaining a positive trans side voltage bias, sequencing of the DNA template with the lambda DNA exonuclease nanosensor is initiated by adding MgCl2, a cofactor necessary for exonuclease activity, to a final concentration of 10 mM in the cis reservoir. Temperature is maintained at 23° C. during the sequencing reaction. A voltage of 80 mV is applied and maintained and conductivity through the membrane is measured over time as the exonuclease processes the template nucleic acid on the exterior of the pore according to its natural functions while undergoing conformational changes that modulate the flow of current through the nanopore.

### Example 5

### Assembly and Use of a Molecular Sensor Complex Based on a DNA Helicase Nucleic Acid Processing Enzyme

This Example describes how a DNA helicase nucleic acid processing enzyme may be assimilated with a nanopore embedded in a lipid bilayer membrane to form a sensor complex for DNA sequencing applications. In this Example, the helicase is the Dab-like helicase, bacteriophage T7 gp4, with inherent duplex strand separation activity. First, a lipid bilayer membrane is formed with the lipid 1,2-diphytanoyl-sn-glycero-3-phosphoethanolamine (C45H90NO8P). Briefly, as described previously, the lipid bilayer is formed over an aperture in a PTFE solid support cell by priming the cell with a thin coat of lipid dissolved in hexane and coating over the support cell. Hexane is removed by air-drying the painted cell and a lipid membrane is painted over the support cell by dissolving PE in 1-hexadecene and depositing the solution over the primed support cell with a pipette and moving an air bubble over the aperture in the support cell to form a lipid bilayer membrane over the aperture. Next, an aqueous solution of the nanopore, e.g., αHL, is added to the lipid bilayer and the pore is allowed to self-assemble in the membrane and insert to form a transmembrane ion conductive pore.

A helicase-DNA template complex is next generated. In this Example, the DNA helicase and double-stranded DNA template are produced using standard molecular biology technologies. In this example, the helicase is modified by covalent attachment of a tether construct, as described in Example 1. The double-stranded DNA template is incubated with the T7 gp4 DNA helicase in an aqueous solution containing 30 mM Tris-HCl, pH 7.5, 10 mM MgCl2 4 mM DTT, and 30 mM ammonium acetate, which binds the DNA template following its natural functions. The T7 gp4 helicase-DNA template assembly is then assimilated, or coupled, with the nanopore embedded in the lipid bilayer membrane by adding the assembly to the cis reservoir of the nanopore sensor containing an aqueous solution of 30 mM Tris-HCl, pH 7.5, 10 mM MgCl2, 4 mM Dithiothreitol, and 300 mM Ammonium Acetate in the cis reservoir and an aqueous solution of 1000 mM NH4Cl on the trans side reservoir. An electric potential is applied across the membrane to thread the negatively charged tether through the pore, thereby guiding and the helicase-template complex to the nanopore. The helicase is secured to the nanopore by hybridizing a short oligonucleotide anchor to the tether construct on the distal side of the nanopore.

While maintaining a positive trans side voltage bias, sequencing of the DNA template with the T7 gp4 DNA helicase-nanopore sensor complex is initiated by adding ATP to the cis reservoir. Temperature is maintained at 23° C. during the sequencing reaction. A voltage of 80 mV is applied and maintained and conductivity through the membrane is measured over time as the helicase processes the template nucleic acid external to the pore according to its natural functions and undergoes conformational changes that modulate the flow of current through the nanopore.

### Example 6

### Assembly and Use of a Low-Noise Solid-State Molecular Sensor Complex Based on a DNA Polymerase Nucleic Acid Processing Enzyme and a Solid-State Chip

This Example describes how a DNA polymerase nucleic acid processing enzyme may be assimilated with a low-noise solid-state support chip to form a sensor complex for DNA sequencing applications. In this Example, the polymerase is the Phi29 DNA polymerase with inherent polynucleotide strand-displacement and exonuclease activities. First, a low capacitive solid-state chip is fabricated starting from a silicon chip with dimensions of 200 μm×10 μm. The chip is cleaned using the RCA process and then the following coatings are applied to the chip: 1) 30 nm LPCVP silicon (Si) lean silicon nitride (SiN) on both sides; 2) 3 μm PECVD SiO2 on the backside of the chip; 3) 200 nm PECVD SiN on the backside of the chip. Lithography masking technology is then used to RIE etch wells of 30 nm into the SiN on the front side of the chip. Lithography masking technology is then further used to RIE etch wells of 200 nm on the on the backside of the chip. Finally, KOH aniso/isotropic etching is used to create the geometry of the chip. The nanopore, 4 nm in diameter, is drilled into the 30 nm thick silicon nitride membrane using a FEI Technai-transmission electron microscope.

A test apparatus has 2 reservoirs filled with electrolyte solution, which are separated by the silicon chip mounted on a gasket so that the only fluid connection between the reservoirs is through the nanopore located in the silicon nitride membrane of the chip. Each reservoir has a Ag/AgCl electrode through which potential is applied and current can be measured with a Molecular Devices Axopatch 200B amplifier.

A polymerase-DNA template complex is generated next. In this Example, the Phi29 DNA polymerase, double-stranded DNA template, and oligonucleotide primers are produced using standard molecular biology technologies. In this example, the polymerase is modified by covalent attachment of a tether construct, as described in Example 1. The double-stranded DNA template is complexed with an appropriate oligonucleotide primer and the primed DNA template is incubated with the Phi29 DNA polymerase in an aqueous solution containing 30 mM Tris-HCl, pH 7.5, 10 mM MgCl2 4 mM DTT, and 30 mM ammonium acetate, which binds the complex following its natural functions. The Phi29 polymerase-DNA template assembly is then assimilated, or coupled, with the solid-state chip by adding the polymerase assembly to the cis reservoir of the test apparatus containing an aqueous solution of 30 mM Tris-HCl, pH 7.5, 10 mM MgCl2, 4 mM Dithiothreitol, and 300 mM Ammonium Acetate in the cis reservoir and an aqueous solution of 1000 mM NH4Cl on the trans side reservoir. An electric potential is applied across the chip to thread the negatively charged tether through the nanopore, thereby guiding and the DNA polymerase-template complex to the nanopore. The polymerase is secured to the pore by hybridizing a short oligonucleotide anchor to the tether construct on the distal side of the nanopore.

While maintaining a positive trans side voltage bias, sequencing of the DNA template with the solid-state nanosensor chip is initiated by adding a mixture of all four deoxyribonucleotide triphosphate substrates to the cis side of the reservoir to a final concentration of 100 μM of each dNTP. Temperature maintained at 20° C. A voltage of 80 mV is applied and maintained and conductivity through the chip is measured over time as the polymerase processes the template nucleic acid according to its natural functions and undergoes conformational changes that modulate the flow of current through the nanopore.

While the disclosed subject matter is described herein in terms of certain embodiments, those skilled in the art will recognize that various modifications and improvements can be made to the application without departing from the scope thereof. Thus, it is intended that the present application include modifications and variations that are within the scope of the appended claims and their equivalents. Moreover, although individual features of one embodiment of the application can be discussed herein or shown in the drawings of one embodiment and not in other embodiments, it should be apparent that individual features of one embodiment can be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the application such that the application should be recognized as also specifically directed to other embodiments having any other possible combinations. Thus, the foregoing description of specific embodiments of the application has been presented for purposes of illustration not description. It is not intended to be exhaustive or to limit the application to those embodiments disclosed.

1. A method for determining sequence information about a nucleic acid molecule, the method comprising the steps of:
providing a membrane having at least one transmembrane pore, the at least one transmembrane pore having a top opening and a bottom opening, and having a single processive nucleic acid processing enzyme localized proximal to one of the openings, the processive nucleic acid processing enzyme complexed with the nucleic acid;
contacting the processive nucleic acid processing enzyme with an ion conductive reaction mixture comprising reagents required for nucleic acid processing by the enzyme;
providing a voltage differential that induces ion current through the pore, wherein the ion current is only substantially-modulated by nucleotide-dependent conformational changes in the processive nucleic acid processing enzyme;
measuring the current through the transmembrane pore over time to detect the nucleotide-dependent conformational changes in the processive nucleic acid processing enzyme; and
identifying the type of nucleotides processed by the processive nucleic acid processing enzyme using current modulation characteristics, thus determining sequence information about the nucleic acid molecule.

2. The method of claim 1 wherein the current modulation characteristics comprise the magnitude of the current through the transmembrane pore.

3. The method of claim 1 wherein the current modulation characteristics comprise the shape of the measured current through the transmembrane pore over time.

4. The method of claim 1 wherein the transmembrane pore comprises a protein.

5. The method of claim 2 wherein the protein is selected from the group consisting of αHL, MspA, and OmpG.

6. The method of claim 5 wherein the polypeptide is OmpG.

7. The method of claim 6 wherein the current modulation characteristics comprise changes to spontaneous OmpG current gating activity.

8. The method of claim 1 wherein the processive nucleic acid processing enzyme is a DNA polymerase.

9. The method of claim 8 wherein the DNA polymerase is selected from the group consisting of Klenow fragment, Phi29, and DPO4.

10. The method of claim 8 wherein the nucleic acid is a primed single stranded template.

11. The method of claim 8 wherein the reaction mixture comprises reagents required for polymerase mediated nucleic acid synthesis.

12. The method of claim 8 wherein the nucleotide-dependent conformational changes are produced by binding of single nucleotides and incorporation into a growing strand by the DNA polymerase.

13. The method of claim 11 wherein the sequencing reaction mixture comprises four different types of nucleotides or nucleotide analogs, each corresponding to the bases A, G, C, and T, or A, C, G, and U.

14. The method of claim 13 wherein each of the types of nucleotides or nucleotide analogs produces a different conformational change in the polymerase enzyme.

15. The method of claim 14 wherein the different conformational changes are structurally distinct.

16. The method of claim 14 wherein the different conformational changes are temporally distinct.

17. The method of claim 15 or 16 wherein the different conformational changes have different current blockage levels.

18. The method of claim 8 wherein the step of contacting the DNA polymerase with an ion conductive reaction mixture comprising reagents required for nucleic acid processing comprises the steps of sequentially flooding the DNA polymerase with mixtures comprising each single nucleotide.

19. The method of claim 1 wherein the processive nucleic acid processing enzyme is a DNA exonuclease.

20. The method of claim 19 wherein the exonuclease is a native or an engineered enzyme with exonuclease activity.

21. The method of claim 19 wherein the nucleic acid is a double-stranded or single-stranded nucleic acid.

22. The method of claim 19 wherein the reaction mixture comprises reagents required for exonuclease mediated nucleic acid degradation.

23. The method of claim 19 wherein the binding and release of single nucleotides from the nucleic acid produce the nucleotide-dependent conformational changes in the exonuclease.

24. The method of claim 23 wherein each type of nucleotide produces a different conformational change in the exonuclease enzyme.

25. The method of claim 24 wherein the different conformational changes are structurally distinct.

26. The method of claim 24 wherein the different conformational changes are temporally distinct.

27. The method of claim 25 or 26 wherein the different conformational changes have different current modulation levels.

28. The method of claim 1 wherein the processive nucleic acid processing enzyme is a DNA helicase.

29. The method of claim 28 wherein the helicase is a native or an engineered enzyme possessing helicase activity.

30. The method of claim 28 wherein the nucleic acid is a double-stranded nucleic acid.

31. The method of claim 28 wherein the reaction mixture comprises reagents required for helicase mediated nucleic acid strand separation.

32. The method of claim 28 wherein the breaking of hydrogen bonds between individual pairs of nucleotides produces the nucleotide-dependent conformational changes in the DNA helicase.

33. The method of claim 32 wherein each type of paired nucleotides produces a different conformational change in the helicase enzyme.

34. The method of claim 33 wherein the different conformational changes are structurally distinct.

35. The method of claim 33 wherein the different conformational changes are temporally distinct.

36. The method of claim 34 or 35 wherein the different conformational changes have different current modulation levels.

37. The method of claim 1 wherein the processive nucleic acid processing enzyme is localized to the top opening of the transmembrane pore.

38. The method of claim 1 wherein the processive nucleic acid processing enzyme is localized to the bottom opening of the transmembrane pore.

39. The method of claim 1 wherein the processive nucleic acid processing enzyme is localized to the transmembrane pore by covalent linkage to a threading tether.

40. The method of claim 39 wherein the threading tether comprises polyethylene glycol (PEG) repeats.

41. The method of claim 40 wherein the length of the PEG repeats is sufficient to span the transmembrane pore channel.

42. The method of claim 40 wherein the threading tether further comprises at least one current modulating substituent disposed within the PEG repeats.

43. The method of claim 41 wherein the threading tether further comprises a molecular anchor disposed at the opening of the transmembrane pore opposite the processive nucleic acid processing enzyme, wherein the molecular anchor secures the tether in place within the pore.

44. The method of claim 43 wherein the molecular anchor is a doubled stranded oligonucleotide or a biotin-streptavidin conjugate.

45. The method of claim 44 wherein the molecular anchor is a double stranded oligonucleotide.

46. The method of claim 39 wherein the threading tether is attached to a stationary domain of the processive nucleic acid processing enzyme.

47. The method of claim 39 wherein the threading tether is attached to a mobile domain of the processive nucleic acid processing enzyme.

48. The method of claim 39 wherein the processive nucleic acid processing enzyme is covalently attached to the transmembrane pore by at least one linker.

49. The method of claim 48 wherein the at least one linker restricts substantial movement of the processive nucleic acid processing enzyme relative to the transmembrane pore.

50. The method of claim 1 wherein the processive nucleic acid processing enzyme is localized to the transmembrane pore by direct covalent linkage between a mobile domain in the enzyme and a position that blocks current flow in the transmembrane pore.

51. The method of claim 1 wherein the processive nucleic acid processing enzyme and the transmembrane pore comprise a fusion protein.

52. The method of claim 1 wherein the processive nucleic acid processing enzyme is disposed within the transmembrane pore.

53. The method of claim 1 wherein the amino acid sequence of the processive nucleic acid processing enzyme is genetically altered to modify the charge of the enzyme at the transmembrane pore interface.

54. The method of claim 1 wherein the amino acid sequence of the processive nucleic acid processing enzyme is genetically altered to optimize enzyme activity in high salt buffers.

55. The method of claim 1 wherein the transmembrane pore comprises at least one current modulating substituent disposed in the interior of the pore.

56. The method of claim 1 wherein the voltage drop is AC or DC.

57. The method of claim 1 wherein the nucleic acid remains external to the pore during processing by the processive nucleic acid processing enzyme.

58. A construct comprising an ion conductive pore and a processive nucleic acid processing enzyme, wherein the ion conductive pore has a top opening and a bottom opening, wherein the enzyme is localized proximal to one of the openings and undergoes conformational changes in response to processing of a nucleic acid external to the pore, and wherein the conformational changes modulate current flow through the pore.

59-80. (canceled)

81. A system for determining the nucleotide sequence of a polynucleotide in a sample, the system comprising:
a cis chamber and a trans chamber, wherein the cis chamber and the trans chamber are separated by a membrane and wherein the cis and trans chamber include an electrically conductive mixture;
a construct according to any one of claims 57-79 assimilated with the membrane to provide a transmembrane pore and a processive nucleic acid processing enzyme, wherein the enzyme undergoes conformational changes in response to processing of the polynucleotide;
a reaction mixture in contact with the processive nucleic acid processing enzyme comprising reagents required for nucleic acid processing by the enzyme;
drive electrodes in contact with the electrically conductive reaction mixture on either side of the membrane for producing a voltage drop across the transmembrane pore;
one or more measurement electrodes connected to electronic measurement equipment for measuring ion current through the transmembrane pore; and
a computer to translate the ion current measurement into nucleic acid sequence information.

82. A method of assembling a molecular sensor complex comprising:
providing a transmembrane pore embedded in a membrane;
delivering a processive nucleic acid processing enzyme-tether conjugate to a first side of the membrane, wherein the tether comprises a pore spanning segment, a first oligonucleotide segment, and a tail segment of substantial negative charge;
applying a voltage bias to the first side of the membrane sufficient to localize the conjugate to the transmembrane pore; and
delivering a second oligonucleotide complementary to the first oligonucleotide segment to a second side of the membrane, wherein the second oligonucleotide hybridizes to the first oligonucleotide segment and secures the processive nucleic acid processing enzyme-tether conjugate to the transmembrane pore.

83-87. (canceled)
